(19) United States
(12) Patent Application Publication
(10) Pub. No.: US 2009/0136481 A1
(43) Pub. Date: May 28, 2009

(54) Use of Myostatin (GDF-8) Antagonists for Treatment of Sarcopenia (Age-Related Muscle-Wasting)

(75) Inventors: Ravi Kambadur, Hamilton (NZ); Mridula Sharma, Hamilton (NZ); Alex Hennebry, Hamilton (NZ); Monica Senna Salerno de Moura, Hamilton (NZ)

Correspondence Address: NIXON & VANDERHYE, PC, 901 NORTH GLEBE ROAD, 11TH FLOOR, ARLINGTON, VA, 22203, US

(73) Assignee: Orico Limited, Dunedin (NZ)

(21) Appl. No.: 11/883,854

(22) PCT Filed: Feb. 7, 2006
(86) PCT No.: PCT/NZ06/00010

§ 371 Date: Nov. 6, 2007

(30) Foreign Application Priority Data
Feb. 7, 2005 (NZ) 538097

(51) Int. Cl. A61K 38/16; A61K 39/395; A61K 38/02; A61P 21/00; A61K 38/18; A61K 31/7088

(52) U.S. Cl. 424/130.1; 514/2; 514/44; 514/12

(57) ABSTRACT

The present invention relates to a method of treating sarcopenia in a human or animal patient by inhibiting the activity of myostatin using one or more myostatin antagonists.

### FIELD OF THE INVENTION

This invention relates to a method of inducing muscle regeneration via activation of satellite cells, particularly, although by no means exclusive, for treating sarcopenia.

### BACKGROUND

The normal mechanism involved in muscle tissue regeneration initially involves the recruitment of satellite cells. Muscle satellite cells are a distinct lineage of myogenic progenitors which are located between the basal lamina and sarcolemma of mature myofibers (Bischoff, 1994; Grounds and Yablonka-Reuveni, 1993). During the regeneration cycle, satellite cells are activated and migrate from the myofibres to the site of regeneration to give myoblasts. Most of the proliferating myoblasts differentiate into myotubes. The myotubes mature and are incorporated into muscle fibres. The remaining myoblasts return to the myfibers to renew the satellite cell population, and thus the capacity to continue the regeneration cycle FIG. 1—schematic).

Recent studies have also demonstrated a role for macrophages during the early events of skeletal muscle regeneration (Merly et al., 1999). A transplantation model showed that stimulation of macrophage infiltration resulted in earlier activation of satellite cells, demonstrating that macrophages indeed play a direct role in muscle regeneration (Lescaudron et al., 1997; Lescaudron et al., 1993).

The muscle regeneration cycle occurs continuously throughout an individuals lifetime when worn out or damaged muscle tissue is replaced. However, as the body ages the muscle regeneration cycle becomes less efficient. Sarcopenia, resulting in a decline in muscle mass and performance, is associated with normal aging. Whilst the skeletal muscle is still capable of regenerating itself, it appears that the environment in old aged muscles is less supportive towards muscle satellite cell activation, proliferation and differentiation, resulting in a net loss of muscle tissue (Greenlund and Nair, 2003).

The nature of the chemical signals that direct the migration of macrophages, satellite cells and myoblasts during skeletal muscle regeneration is not fully understood.

Some growth factors, including Hepatocyte Growth Factor (HGF), Fibroblast Growth Factor (FF) and Mechano Growth Factor (MGF), have been shown to positively affect muscle regeneration by regulating satellite cell activation (Floss et al., 1997; Miller et al., 2000, Goldspink and Harridge; 2004). However, presently, no growth factors are in clinical use and treatment of sacropenia is limited to physical exercise, or growth hormone supplementation (Greenlund and Nair, 2003). These therapies have met with limited success.

There is thus a need to provide an effective clinical treatment for muscle regeneration via satellite cell activation proliferation and differentiation in sarcopenia.

It is an object of the present invention is to go someway towards fulfilling this need and/or to at least provide a useful choice.

### SUMMARY OF THE INVENTION

Surprisingly, the growth factor myostatin, a member of the TGF-beta family of growth factors, has been shown for the first time to be implicated in the etiology of sarcopenia. Inhibition of myostatin activity has been found to significantly improve the activation of satellite cells in an animal model of sarcopenia.

Accordingly, the present invention provides a method of treating sarcopenia comprising the step of administering an effective amount of at least one myostatin antagonist to a patient in need thereof. The invention may be useful in treating sarcopenia both humans and non-human patients, as well as sarcopenia related diseases which are characterised by muscle atrophy and a decrease in the ability of satellite cells to become activated.

The myostatin antagonist may be selected from any one or more known myostatin inhibitors. For example, U.S. Pat. No. 6,096,506 and U.S. Pat. No. 6,468,535 disclose anti-myostatin antibodies. U.S. Pat. No. 6,369,201 and WO 01/05820 teach myostatin peptide immunogens, myostatin multimers and myostatin immunoconjugates capable of eliciting an immune response and blocking myostatin activity. Protein inhibitors of myostatin are disclosed in WO 02/085306, which include the truncated Activin type II receptor, the myostatin pro-domain, and follistatin. Other myostatin inhibitors derived from the myostatin peptide are known, and include for example myostatin inhibitors that are released into culture from cells overexpressing myostatin (WO 00/43781); dominant negatives of myostatin (WO 01/53350), which include the Piedmontese allele (cysteine at position 313 is replaced with a tyrosine) and mature myostatin peptides having a C-terminal truncation at a position either at or between amino acid positions 335 to 375. US200410181033 also teaches small peptides comprising the amino acid sequence WMCPP, and which are capable of binding to and inhibiting myostatin.

Preferably, the one or more myostatin antagonists comprise one or more dominant negatives selected from the group consisting of myostatin peptides that are C-terminally truncated at a position at or between amino acids 335, 350 and the Piedmontese allele.

The one or more myostatin antagonists may also include a myostatin splice variant comprising a polypeptide of any one of SEQ ID Nos: 8-14 or a functional fragment or variant thereof, or a sequence having 95%, 90% 85%, 80%, 75% or 70% sequence identity thereto.

The one or more myostatin antagonists may also include a regulator involved in the myostatin pathway comprising a polypeptide of SEQ ID No. 16 or SEQ ID No. 18, or a functional fragment or variant thereof, or a sequence having at least 95%, 90%, 85%, 80%, 75% or 70% sequence identity thereto.

The myostatin antagonist may also include an anti-sense polynucleotide, an interfering RNA molecule, for example RNAi or siRNA, or an anti-myostatin ribozyme, which would inhibit myostatin activity by inhibiting myostatin gene expression.

When the one or more myostatin antagonists include an antibody, the antibody may be a mammalian or non-mammalian derived antibody, for example an IgNAR antibody derived from sharks, or the antibody may be a humanised antibody, or comprise a functional fragment derived from an antibody.

The present invention also provides for the use of one or more myostatin antagonists in the manufacture of a medicament for treating sarcopenia in a patient in need thereof.

The one or more myostatin antagonists may be selected from the group of myostatin antagonists disclosed above.

The medicament may be formulated for local or systemic administration, for example, the medicament way be formulated for injection directly into a muscle, or may be formulated for oral administration for systemic delivery to the muscle.

The present invention further provides a composition comprising one or more myostatin antagonists together with a pharmaceutically acceptable carrier, for use in the treatment of sarcopenia in a patient in need thereof.

The present invention further provides one or more myostatin antagonists for use in the treatment of sarcopenia in a patient in need thereof.

The invention will now be described in more detail with reference to the figures of the accompanying drawings in which:

### BRIEF DESCRIPTION OF THE FIGURES

FIG. 1 shows a schematic model for the role of satellite cells in muscle regeneration;

FIG. 2A shows inhibition of satellite cell activation by myostatin;

FIG. 2B shows that inhibition of satellite cells activation by myostatin is reversible when myostatin is removed from the media (Rescue);

FIG. 2C shows the effect of myostatin on the migration of satellite cells;

FIG. 2D shows a photomicrograph of a typical myofiber with BrdU positive nuclei (i) and the same myofiber with DAPI stained nuclei, (ii);

FIG. 3A shows the percent of satellite cells per 100 myonuclei, on fibers isolated from 1 and 24 month old wild-type and nyostatin-null TA muscle. Satellite cells were visualized by immunostaining for CD34 and total nuclei by DAPI counterstaining. Fibers were isolated from 3 animals per group and in excess of 1,000 nuclei per group were counted (P<0.001);

FIG. 3B shows the percent of activated satellite cells per 100 myonuclei, on fibers isolated from 1 and 24 month old wild-type and myostatin-null TA muscle. Activated satellite cells were represented by in vitro BrdU incorporation and total nuclei by DAPI counterstaining. Fibers were isolated from 3 animals per group and over 1,000 nuclei per group were counted (P<0.05);

FIG. 3C shows the percent of BrdU positive cells determined through flow cytometry. Satellite cells were BrdU labelled in vivo and isolated from 1 and 6 month old wild-type and myostatin-null hind limb muscle using a Percoll gradient. A minimal of 10,000 cells per sample group were analysed in triplicate (P<0.001). Empty bars representative of 1 month old mice, solid bars representative of 6 month old mice. Different lower case letters indicate significant differences between data;

FIG. 4 shows the number of PCNA positive nuclei on isolated fibres. Isolated fibres were incubated with 5 or 10 μg of 350 and immunostained with PCNA antibodies to determine the number of activated satellites cells per 100 myonuclei. Data are expressed as mean±s.e.m (**=P<0.001;

FIG. 5A shows hematoxylin and eosin staining of control muscle sections from wild type and myostatin null mice;

FIG. 5B shows a low power view one day (D1) after notexin injection;

FIG. 5C shows a higher power view of the same sections as (B) stained to show eosinophilic (e) cytoplasm and fine intracellular vacuolation (v) of the myofibers with an increase in the intracellular spaces and marked myofiber disruption (arrows);

FIG. 5D shows day 2 (D2) muscle sections, with increased numbers of nuclei in muscle of myostatin null mice (arrows). Arrow heads denote the myonuclei along the margins of the necrotic myofibers;

FIG. 5E shows day 3 (D3) muscle sections with infiltrating mononucleated cells in both wild type and myostatin null muscle, but with higher numbers in the myostatin null sections. The scale bar equals 10 μm;

FIG. 5F shows day 5 sections (D5), having an increased number of nuclei in notexin treated myostatin null muscle sections;

FIG. 6A shows the percentage of MyoD positive myogenic precursor cells in wild type (Mstn^{+/+}) and myostatin null (Mstn^{-/-}) regenerating muscle;

FIG. 6B shows the percentage of Mac-1 positive cells in wild type (Mstn^{+/+}) and myostatin null (Mstn^{-/-}) regenerating muscle;

FIG. 6C shows the expression profiles of MyoD and myogenin genes in control uninjured muscle (C) and regenerating wild type (wt) and myostatin null (Mstn null) muscle up to 28 days after notexin injection. GAPDH was used as a control to show equal amount of RNA used;

FIG. 7 shows the average number of Mac1 positive cells in regenerated muscle 2, 3, 7 and 10 days after notexin injection in saline treated and myostatin inhibitor 350 treated mice;

FIG. 8 shows immunofluorescence on tissue sections obtained from myostatin knock-out (KO) and wild-type (WT) mice at day 14 (D14), 21(D21) and 28(D28) after notexin injection. WT tissue show stronger intensity of staining i.e. a higher concentration of vimentin positive cells when compared with KO tissue;

FIG. 9 shows the chemo-inhibitory effect of myostatin on macrophage migration and recovery using a myostatin antagonist (dominant negative myostatin peptide C-terminally truncated at amino acid 350);

FIG. 10A shows the chemo-attractant effect of myostatin on ovine primary fibroblast;

FIG. 10B shows the chemo-inhibitory effect of myostatin on ovine primary myoblasts and recovery using a myostatin antagonist (dominant negative myostatin peptide C-terminally truncated at amino acid 350);

FIG. 11 shows photomicrographs low power (i) and high power (ii) of Hematoxylin and eosin staining (H&E) and Van Geisen (iii) staining of day 28 (D28) wild type and myostatin null muscle sections. Thick connective tissue (arrows) is seen in wild type muscle sections (ii); collagen (arrows) is seen in the wild type muscle sections (iii), scale bar equals 10 μm; a scanning electron micrograph of wild type and myostatin null muscle is shown in (iv) after 24 days of regeneration; scale bar equals 120 μm;

FIG. 12 shows the effect on muscle weight of a myostatin antagonist (dominant negative myostatin peptide C-terminally truncated at amino acid 350) in mice recovering from notexin injection;

FIGS. 13A-D show hematoxylin and eosin staining of muscle sections from regenerating muscle after notexin injection at day 7 (A-saline treated; B-myostatin antagonist 350 treated) and at day 10 (C-saline treated; D-myostatin antagonist 350 treated). Asterisks show necrotic areas;

FIG. 14 shows the percentage of unregenerated □ and regenerated [symbol] areas of the muscle sections of FIG. 13;

FIG. 15 shows the percentage of collagen formation in regenerating muscle 10 and 28 days after notexin injection in saline treated and myostatin inhibitor 350 treated mice;

FIG. 16 shows the average fibre area of regenerated muscle fibres 28 days after notexin injection in saline treated and myostatin inhibitor 350 treated mice;

FIG. 17 shows Gene Pax7 (A) and MyoD (B) protein levels (detected through western blotting) 1, 3, 7, 10 and 28 days after the administration of notexin in saline (sal) and 350 treated TA muscles; and

FIG. 18 shows an increased inflammatory response in regenerating muscle 2 and 4 days after damage and an increased muscle mass in regenerated muscle (at 21 days).

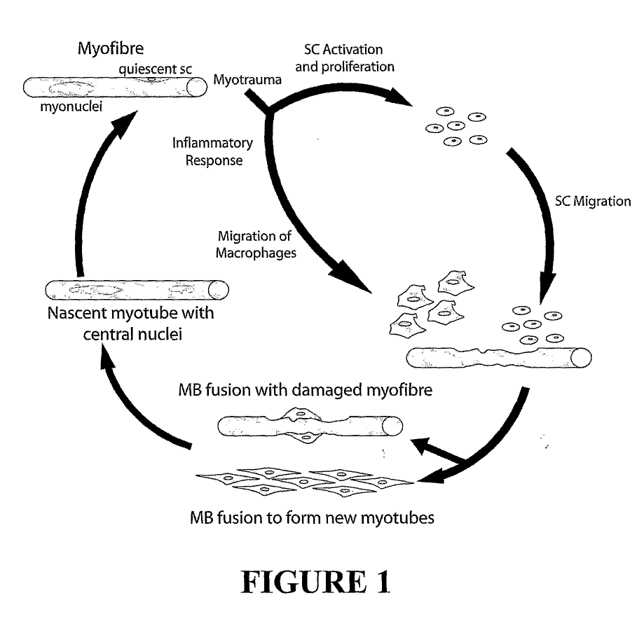

### DEFINITIONS

“Sarcopenia” as used throughout the specification and claims means a decline in muscle mass and performance caused by old age, as well as sarcopenia related conditions characterised by muscle atrophy and a decrease in the ability of satellite cells to become activated.

“Hypertrophy” as used throughout the specification and claims means any increase in cell size.

“Hyperplasia” as used throughout the specification and claims mean any increase in cell number.

“Muscle atrophy” as used throughout the specification and claims means any wasting or loss of muscle tissue resulting from the lack of use.

“Inhibitor” or “antagonist” as used throughout the specification and claims means any compound that acts to decrease, either in whole or in part, the activity of a protein. This includes a compound that either binds to and directly inhibits that activity of the protein, or may act to decrease the production of the protein or increase its production, thereby affecting the amount of the protein present and thereby decreasing its activity.

“Gene expression” as used through the specification and claims means the initiation of transcription, the transcription of a section of DNA into mRNA, and the translation of the mRNA into a polypeptide.

“Comprising” as used throughout the specification and claims means ‘consisting at least in part of’, that is to say when interpreting independent claims including that term, the features prefaced by that term in each claim all need to be present but other features can also be present.

### DETAILED DESCRIPTION OF THE INVENTION

The present invention shows for the first time that myostatin is involved in the etiology of sarcopenia. In particular, myostatin appears to be a negative regulator of satellite cell activation, proliferation and differentiation and thus muscle regeneration in sarcopenia and in sarcopenia related diseases characterised by skeletal muscle atrophy and a decrease in the ability of satellite cells to become activated.

Myostatin is a known growth factor involved in regulation of muscle growth. In particular, myostatin is a member of the TGF-β family of growth factor and is a potent negative regulator of myogenesis (McPherron et al., 1997).

Knock-out mice for myostatin have greatly increased muscle mass over their entire body. Myostatin-null mice have approximately 30% greater body weight than normal mice, and exhibit a 2-3-fold increase in individual muscle weights due to muscle fibre hyperplasia and hypertrophy. Natural mutations in myostatin have been identified as being responsible for the “double-muscled” phenotype, such as the Belgian Blue and Piedmontese cattle breeds (McPherron et al 1997b, Kambadur et al. 1997, Grobet et al. 1997).

Recent studies suggest that myostatin is a potent regulator of cell cycle progression and function by regulating both the proliferation and differentiation steps of myogenesis Langley et al., 2002; Thomas et al., 2000). Several studies have demonstrated a role for myostatin not only during embryonic myogenesis, but also in postnatal muscle growth. Studies by Wehling et al (Wehling et al., 2000) and Carlson et al (Carlson et al., 1999) indicated that atrophy-related muscle loss due to hind limb suspension in mice was associated with increased myostatin levels in M. plantaris. Increased myostatin levels were also associated with severe muscle wasting seen in HIV patients (Gonzalez-Cadavid et al., 1998). One explanation for the elevated levels of myostatin observed during muscle disuse is that myostatin may function as an inhibitor of satellite cell activation. Indeed this is supported by recent studies which show that a lack of myostatin results in an increased pool of activated satellite cells in vivo and enhanced self-renewal of satellite cells (McCroskery et al., 2003).

To date many potential uses of myostatin have been suggested including the development of myostatin inhibitors to help regulate the overall body mass of an animal, or for use in treating conditions associated with generalized muscle wasting. However, currently there are no myostatin inhibitors that are in clinical or veterinary use. In addition, mystatin has not previously been linked to the natural decline in muscle mass and function seen in aging (sarcopenia).

The present invention is thus directed to a method of treating sarcopenia comprising the step of administering an effective amount of at least one myostatin antagonist to a patient in need thereof. The patient is preferably a human patient, but the method of the present invention may also be used to treat sarcopenia in non-human animals.

The myostatin antagonist may be selected from one or more molecules that are capable of inhibiting, in whole or in part, the activity of myostatin.

In particular, myostatin antagonist may be selected from any one or more known myostatin inhibitors. For example, U.S. Pat. No. 6,096,506 and U.S. Pat. No. 6,468,535 disclose anti-myostatin antibodies. U.S. Pat. No. 6,369,201 and WO 01/05820 teach myostatin peptide immunogens, myostatin multimers and myostatin immunoconjugates capable of eliciting an immune response and blocking myostatin activity. Protein inhibitors of myostatin are disclosed in WO 02/085306, which include the truncated Activin type II receptor, the myostatin pro-domain, and follistatin. Other myostatin inhibitors derived from the myostatin peptide are known, and include for example myostatin inhibitors that are released into culture from cells overexpressing myostatin (WO 00/43781); dominant negatives of myostatin (WO 01/53350), which include the Piedmontese allele (cysteine at position 313 is replaced with a tyrosine) and mature myostatin peptides having a C-terminal truncation at a position either at or between amino acid positions 335 to 375. US2004/0181033 also teaches small peptides comprising the amino acid sequence WMCPP, and which are capable of binding to and inhibiting myostatin.

Preferably, the myostatin antagonist is a dominant negative peptide. These are peptides derived from a parent protein that act to inhibit the biological activity of the parent protein. As mentioned above, dominant negative peptides of myostatin are known and include a mature myostatin peptide that is C-terminally truncated at a position at or between amino acids 335, 350 and the Piedmontese allele (wherein the cysteine at position 3 is replaced with a tyrosine).

Myostatin is initially produced as a 375 amino acid precursor molecule having a secretary signal sequence at the N-terminus, which is cleaved off to leave an inactive pro-form. Myostatin is activated by furin endoprotease cleavage at Arg 266 releasing the N-terminal pro-domain (or latency-associated peptide LAP) domain) and the mature myostatin domain. However, after cleavage, the pro-domain can remain bound to the mature domain in an inactive complex (Lee et al 2001). Therefore, the pro-domain, or fragments thereof, can also be used in the present invention as a myostatin antagonist to treat sarcopenia.

A splice variant of myostatin has been identified which also acts as a myostatin antagonist (PCT/NZ2005/000250). The myostatin splice variant (MSV) results from an extra splice event which removes a large portion of the third exon. The resulting MSV polypeptide, ovine (oMSV; SEQ ID No: 8) and bovine MSV (bMSV; SEQ ID No: 11) shares the first 257 amino acids with native myostatin propeptide, but has a unique 64 amino acid C-terminal end (ovine oMSV65, SEQ ID No: 9 and bovine bMSV65, SEQ ID No: 12). The mRNA differs by 195 nucleotides, however, the valine residue at position 257 in MSV is the same as the canonical myostatin sequence. The MSV of the Belgian Blue cattle (bMSVbb; SEQ BD No: 7) encodes for a 7aa shorter 314aa protein (SEQ ID No: 14) but the rest of the protein sequence shows complete homology in the two breeds examined. The unique 65aa C-terminal peptide (SEQ ID No: 12) is conserved in bMSVbb

It has also been discovered that a (KERK) cleavage site, for propeptide convertase (PC1-7) which includes furin endopeptidase, exists at position 271 to 274. Cleavage at position 274, releases a 47 amino acid C-terminal mature MSV fragment (ovine oMSV47, SEQ ID No: 10 and bovine bMSV47, SEQ D) No: 13).

The 65 amino acid MSV fragment (SEQ ID NO: 12) has been shown to act as a myostatin antagonist in vitro (PCT/NZ2005/000250) and it is expected that MSV in vivo will act to regulate myostatin activity. Therefore, the MSV polypeptides disclosed herein could be used to inhibit myostatin the therefore treat sarcopenia according to the present invention.

Another myostatin antagonist is a modulator of myostatin gene expression. The myostatin gene expression may be altered by introducing polynucleotides that interfere with transcription and/or translation. For example, anti-sense polynucleotides could be introduced, which may include; an anti-sense expression vector, anti-sense oligodeoxyribonucleotides, anti-sense phosphorothioate oligodeoxyribonucleotides, anti-sense oligoribonucleotides, anti-sense phosphorothioate oligonucleotides, or any other means that is known in the art, which includes the use of chemical modifications to enhance the efficiency of anti-sense polynucleotides. Antisense molecules of myostatin may be produced by methods known in the art such as described in (Rayburn et al 2005) and by knowledge of the myostatin gene sequence (McPherron et al 1997).

It will be appreciated that any anti-sense polypeptide need not be 100% complementary to the polynucleotides in question, but only needs to have sufficient identity to allow the anti-sense polynucleotide to bind to the gene, or mRNA to disrupt gene expression, without substantially disrupting the expression of other genes. It will also be understood that polynucleotides that are complementary to the gene, including 5′ untranslated regions may also be used to disrupt translation of the myostatin protein. Likewise, these complementary polynucleotides need not be 100% complementary, but be sufficient to bind the mRNA and disrupt translation, without substantially disrupting the translation of other genes.

The modulation of gene expression may also comprise the use of an interfering RNA molecule including RNA interference (RNAi) or small interfering RNA (siRNA), as would be appreciated by a skilled worker by following known techniques (Ren et al 2006).

Modulation of gene expression may also be achieved by the use of catalytic RNA molecules or ribozymes. It is known in the art that such ribozymes can be designed to pair with a specifically targeted RNA molecule. The ribozymes bind to and cleave the targeted RNA (Nakamura et al 2005).

Any other techniques known in the art of regulating gene expression and RNA processing can also be used to regulate myostatin gene expression.

A further antagonist of myostatin is a peptide derived from myostatin receptors. Such, receptor derived fragments generally include the myostatin binding domain, which then binds to and inhibits wildtype myostatin. The myostatin receptor is activin type IIB and its peptide sequence is described in (Lee et al 2001). Thus, a skilled worker could produce such receptor antagonists without undue experimentation.

Another myostatin antagonist includes an anti-myostatin antibody. Antibodies against myostatin are known in the art, as described above, as are methods for producing such antibodies. The antibody may be a mammalian or a non-mammalian antibody, for example the IgNAR class of antibodies from sharks; or a fragment or derivative derived from any such protein that is able to bind to myostatin.

It will be appreciated that other molecules involved in the myostatin signalling pathway will be suitable for use in the present invention, particularly molecules that have an antagonistic action to myostatin. One such peptide, known as “mighty”, disclosed in PCT/NZ2004/000308, acts to promote muscle growth. “Mighty” expression is repressed by myostatin and therefore is involved in the same signalling pathway. Therefore it will be appreciated that instead of directly inhibiting myostatin, a peptide which opposes the signalling action of myostatin, for example “mighty”, could be used to treat sarcopenia.

It is anticipated that a polynucleotide that encodes the “mighty” gene (ovine; SEQ ID No: 15 and bovine; SEQ ID No: 17) could be used for localised gene therapy at the muscle site, having either permanent or transient expression of “mighty”, or alternatively the “mighty” protein (ovine; SEQ ID No. 16 and bovine; SEQ ID No. 18) could be used directly. It will be appreciated that due to the redundancy in the genetic code sequences that have essentially the same activity can be produced that are not identical to those disclosed in SEQ ID Nos: 15-18. Furthermore peptides having changes in none critical domains that have the same essential function can also be created. Changes can include insertions, deletions, or changes of one amino acid residue to another. Such variations are encompassed within the scope of the present invention.

The present invention is based on the finding that a myostatin antagonist is able to treat sarcopenia, and therefore any myostatin antagonist, known or developed, is suitable for use in the method. This includes any molecule capable of binding to myostatin, for example, a IMM7 immunity protein from E. coli, or any other class of binding protein known in the art. Other peptides that can bind and inhibit myostatin are know, for example, peptides containing the amino acids WMCPP (US2004/0181033). It will be appreciated that any compound that is capable of inhibiting myostatin will be useful in the method and medicaments of the present invention.

The myostatin antagonists, useful in the method of the present invention, may be tested for biological activity in an animal model or in vitro model of muscle regeneration including sarcopenia as discussed below and suitably active compounds formulated into pharmaceutical compositions. The pharmaceutical compositions of the present invention may comprise, in addition to one or more myostatin antagonists described herein, a pharmaceutically acceptable excipient, carrier, buffer, stabiliser or other material well known in the art. Such materials should be non-toxic and should not interfere with the efficacy of the active ingredient. The precise nature of the carrier or other material will be dependent upon the desired nature of the pharmaceutical composition, and the route of administration e.g. oral, intravenous, cutaneous, subcutaneous, intradermal, topical, nasal, pulmonary, intramuscular or intraperitoneal.

Pharmaceutical compositions for oral administration may be in tablet, lozenge, capsule, powder, granule or liquid form. A tablet or other solid oral dosage form will usually include a solid carrier such as gelatine, starch, mannitol, crystalline cellulose, or other inert materials generally used in pharmaceutical manufacture. Similarly, liquid pharmaceutical compositions such as a syrup or emulsion, will generally include a liquid carrier such as water, petroleum, animal or vegetable oils, mineral oil or synthetic oil.

For intravenous, cutaneous, subcutaneous, intradermal or intraperitoneal injection, the active ingredient will be in the form of a parenterally acceptable aqueous solution which is pyrogen-free and has suitable pH, isotonicity and stability.

For nasal or pulmonary administration, the active ingredients will be in the form of a fine powder or a solution or suspension suitable for inhalation. Alternatively, the active ingredients may be in a form suitable for direct application to the nasal mucosa such as an ointment or cream, nasal spray, nasal drops or an aerosol.

The ability of one or more myostatin antagonists to treat sarcopenia can be demonstrated in an aged mouse model according to the method of Kirk (2000).

In a further embodiment, the invention contemplates the use of one or more muscle growth factors which may be co-administered with the pharmaceutical composition of the present invention to give an additive or synergistic effect to the treatment regime. Such growth factors may be selected from the group consisting of HGF, FGF, IGF, MOF, growth hormone etc. Such substances may be administered either separately, sequentially or simultaneously with at least one myostatin antagonist described herein.

Administration of the pharmaceutical composition of the invention is preferably in a “prophylactically effective amount” or a “therapeutically effective amount”, this being sufficient to show benefit to the individual. The actual amount administered, and rate and time-course of administration, will depend on the nature and severity of the sarcopenia that is being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16^{th} edition, Oslo, A. (ed), 1980.

The present invention is also directed to the use of one or more myostatin inhibitors in the manufacture of a medicament for treating sarcopenia in a patient in need thereof. The one or more myostatin antagonists may be selected from the group of myostatin antagonists described above.

The medicament may be formulated for local or systemic administration, for example, the medicament may be formulated for injection directly into a muscle, or may be formulated for oral administration for systemic delivery to the muscle.

The medicament may further comprise one or more additional muscle growth promoting compounds to give an additive or synergistic effect on treating sarcopneia, selected from the group consisting of HOF, FGF, IGF, MGF, growth hormone etc. The medicament may be formulated for separate, sequential or simultaneous administration of the one or more myostatin antagonists and the one or more muscle growth promoting compounds.

Without being bound by theory, it is thought that myostatin antagonists are effective in treating sarcopenia by inducing satellite cell activation, proliferation and differentiation.

For example, inhibition of myostatin activity, has been shown to have a direct effect on muscle regeneration. In particular, satellite cell and myoblast migration is increased when myostatin is either absent (in myostatin null mice), or is inhibited using a myostatin antagonist. In addition, satellite cell activation has shown to be significantly increased in aged muscle for the first time.

In addition, inhibition of myostatin activity is shown for the first time to have a direct effect on macrophage recruitment. In particular, both the number of macrophages and the migration time to the regeneration site are increased when myostatin is either absent (in myostatin null mice), or is inhibited, using a myostatin antagonist. As discussed above, macrophages are thought to be involved in satellite cell activation.

Thus, it appears that inhibition of myostatin acts both directly, to increase satellite cell migration and activation, as well as acting indirectly on satellite cell activation via macrophage recruitment.

The results in myostatin null mice show indirectly that inhibition of myostatin activity results in increased satellite cell activation, proliferation and differentiation. This suggests that inhibition of myostatin may be useful in increasing satellite cell activation in animals with normal myostatin levels. However, as satellite cells are embryonic in origin and myostatin null mice have a significantly higher population of satellite cells at the embryonic stage, the myostatin null phenotype would not be able to be replicated in a wild-type animal. This is not only because the actual number of satellite cells could not be increased to the myostatin null base level, but also because the muscle cell regeneration cycle per se is more efficient in myostatin null mice. In addition, as myostatin is found in tissues other than muscles, partially knocking out myostatin activity may have adverse side effects. Thus, the effect of inhibiting myostatin activity by the use of myostatin antagonists on the post-natal muscle regeneration cycle in old age is difficult to predict. This is supported by Goldspink and Harridge, 2004, which notes that a suggested therapy for treating sarcopenia would not be to partially knock out myostatin because this would result in impaired respiratory and cardiovascular function. However, surprisingly, the present invention has found for the first time that myostatin antagonists can be used to successfully treat sarcopenia without adverse side effects.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth

The invention consists in the foregoing and also envisages constructions of which the following gives examples only.

### EXAMPLES

### Example 1

### Myostatin Regulates Satellite Cell Activation

### Methods

### In Vivo BrdU Labelling of Satellite Cells

Satellite cell activation was investigated by in vivo 5-bromo-2′-deoxy-uridine (BrdU) labelling. Wild-type and myostatin-null mice were intraperitoneally injected with BrdU (Roche) (30 mg/kg) as a single pulse 2 hours before euthanizing. Satellite cells were isolated following an adapted protocol of Yablonka-Reuveni and Nameroff (1987). Briefly, 1 and 6 month old wild-type and myostatin-null mice (n=10 per group) were killed by CO₂ gas followed by cervical dislocation. Hind limb muscle were dissected out, minced and digested in 0.2% (w/v) type 1A collagenase (>260 CDU/mg, Sigma) in Dulbecco's modified Eagle medium (DMEM) (Invitrogen) for 90 minutes at 37° C. The muscle slurry was triturated then passed through a 70 μM filter (BD Biosciences) before loading onto 70% and 40% Percoll gradients (Sigma) and centrifuged at 2000×g for 20 minutes at 25° C. The interface between the two gradient solutions was recovered and cells were resuspended in PBS. In order to detect BrdU incorporation an In Situ Cell Proliferation Kit, FLUOS (Roche) was used. Cells were fixed for 30 minutes in 70% ethanol on ice and treated with 2N HCL+0.5% TritonX-100 for 30 minutes at room temperature (RT) before neutralising in 0.1 M disodium tetraborate buffer (pH 8.5). Cells were permeabilised in 0.5% Tween-20 in PBS and incubated for 45 minutes with monoclonal anti-BrdU-FLUOS antibody (1:25, Roche) in incubation buffer (Roche) at 37° C. Cells were analyzed by a FACScan (Beckton-Dickinson) flow cytometer.

### Single Myofibre Isolation and Culture

Single fibres were isolated as previously described Rosenblatt et al., (1995). Briefly, 1 and 24 month old wild-type and myostatin-null mice were euthanized by CO₂ gas followed by cervical dislocation. TA were dissected out and digested in 0.2% (w/v) type 1A collagenase (>260 CDU/mg, Sigma) in Dulbecco's modified Eagle medium (DMEM) (Invitrogen) for 60 minutes at 37° C. Muscles were transferred to DMEM+10% horse serum (HS)+0.5% chicken embryo extract (CEE) and fibres were separated by gentle trituration. Isolated fibres were transferred to 4 well chamber slides (Becton Dickinson) coated with 10% matrigel (Becton Dickinson) and either fixed at 37° C. for 10 minutes in 4% paraformaldehyde in PBS or cultured in DMEM+10% HS+0.5% CEE+BrdU at 1:1000 (Roche) for 48 hours at 37° C. in 5% CO₂.

In order to determine the effect of a myostatin antagonist (a dominant negative peptide of myostatin C-terminally truncated at amino acid 350, hereinafter referred to as “350” or “350 protein”) on satellite cell activation, single muscle fibres from TA muscle of 6 months old wild type mice were cultured in presence of either 5 μg/ml or 10 μ/ml 350 in culture media for 32 hour and fixed with methanol and washed in PBS. The fixed fibres were incubated with 1:50 dilution of anti-PCNA antibodies in 0.35% carrageenan lambda overnight. Primary antibody was detected using goat anti-mouse-alexa fluor546. PCNA positive activated satellite cells were counted under microscope and expressed as a percent of total myonuclei.

Satellite cells were detected with CD34 antibodies according to an adapted method of Beauchamp et al., (2000). Briefly, fibres were fixed with paraformaldehyde, washed in PBS, permeablised in 0.5% TritonX-100 in PBS for 10 minutes and blocked in 10% normal goat serum in PBS for 30 minutes at RT. Rat anti-mouse CD34 monoclonal antibody (clone RAM34; PharMingen) at 1:100 in 0.35% carrageenan lambda (Sigma) in PBS was introduced overnight. Primary antibody was detected using biotinylated goat anti-rat IgG polyclonal L antibody (Amersham) at 1:300 in 0.35% carrageenan lambda (Sigma) in PBS for 2 hours at RT followed by streptavidin conjugated Alexa Fluor 488 (Molecular Probes) at 1:400 in 0.35% carrageenan lambda (Sigma) in PBS for 1 hour at RT. Fibres were counterstained with DAPI at 1:1000 in PBS for 5 minutes before mounting with fluorescent mounting medium (Dako) and examining using an Olympus BX50 microscope and SPOT RT camera and software.

To detect BrdU incorporated cells, the 5-bromo-2′-deoxy-uridine labelling and detection kit (Roche) protocol was followed. Fibres were counterstained with DAPI at 1:1000 in PBS for 5 minutes before mounting with fluorescent mounting medium (Dako) and examining using an Olympus BX50 microscope and SPOT RT camera and software.

### Inhibition of Satellite Cell Activation by Myostatin.

Single muscle fibres were isolated from 4 week old wild type mice (n=3) as mentioned above. Fibres were left to attach for 3 min, then 500 μl of fibre media (FM) [DMEM, 10% (v/v) horse serum (HS), 0.5% (v/v) chick embryo extract (CEE), (Penicillin/Streptomycin)] or FM with increasing amounts of recombinant myostatin (Thomas et al., 2000) was added. Purification of recombinant myostatin from E. coli a is described elsewhere (Thomas et al., 2000). Cells were left to migrate off the fibres, for 72 hours at 37° C./5% CO₂. Number of migrated satellite cell in each well was counted under an inverted microscope. Replicates of at least 30 single fibres were used for statistical analysis. Differences between groups were analyzed by a generalized linear model with binomial distribution using GenStat6.

### In Vitro BrdU Incorporation in Activated SC on Fibres:

The muscle fibres were isolated from 4 week old wild type mice (n=6) by the method described above, and allowed to attach to 10% Matrigel coated 4-well Lab-Tek® chamber slides. FM media including 10 μM BrdU with or without increasing concentrations of myostatin was added to the wells and fibres were incubated for 48 hours. In the rescue experiment, isolated fibres were cultured in FM containing 1 μg/ml myostatin for 24 hours and then half were gently washed and changed to FM, while the other half were left in the media with recombinant myostatin for a further 24 hours. Fibres were fixed with Carnoys fixative overnight at −20° C. BrdU incorporation and detection was carried out using the Roche (Roche Diagnostics Corporation International) cell proliferation kit 1 protocol. DAPI staining was used to visualize all myonuclei. BrdU positive nuclei on the fibres (n=30) were counted and the number of BrdU positive nuclei per 100 DAPI positive nuclei were calculated. Differences between groups were analyzed by a generalized linear model with Poisson distribution using GenStat6.

### Results

### Myostatin Inhibits Activation of Satellite Cells

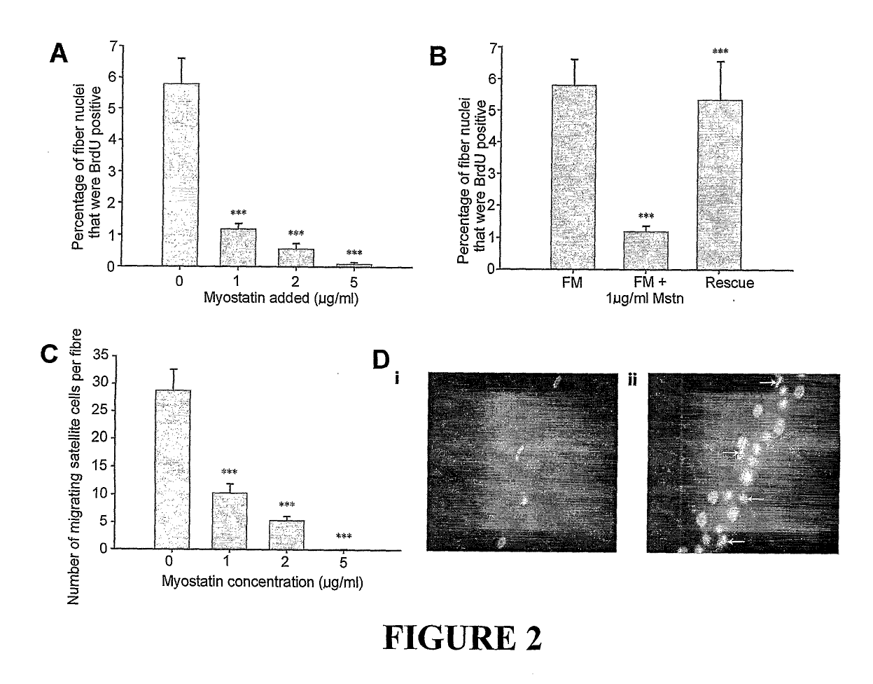

To demonstrate a direct effect of myostatin on satellite cell activation, we assessed satellite cell proliferation after myostatin treatment. Individual muscle fibres isolated from wild type mice were cultured to allow satellite cell activation and proliferation as indicated by BrdU incorporation (Conboy and Rando, 2002; Rosenblatt et al., 1995). In the absence of recombinant myostatin, there was proliferation of satellite cells leading to incorporation of BrdU in 6% of nuclei counted. However, when recombinant myostatin was added to the media in increasing concentrations, fewer satellite cells were proliferating. At 5 μg/ml concentration of myostatin, less than 1% of counted nuclei incorporated BrdU (P<0.001). To prove that the effect of myostatin on satellite cell proliferation was reversible, added recombinant myostatin was removed and upon removal of recombinant myostatin, significantly higher number of satellite cells were proliferating (P<0.001, FIGS. 2 A and B).

These results indicate that myostatin directly inhibits the entry of quiescent satellite cells into the cell cycle. To further study the effect of myostatin on satellite cell proliferation, satellite cells were allowed to detach from fibres to migrate and subsequently proliferate. FIG. 2C demonstrates that on average 30 myoblasts were detected when no recombinant myostatin was added to the culture media. However, the number of migrated myoblasts decreased with increasing concentration of mystatin. These results clearly demonstrate that myostatin directly inhibits the activation of satellite cells

### Effect of Myostatin on Satellite Cell Number and Activation During Ageing.

Myostatin is expressed in satellite cells and a study using young myostatin null mice have shown a lack of myostatin leads to a greater number of satellite cells per unit fibre length as well as an increase in their propensity to become activated (McCroskery et al., 2003). To elucidate the effects of myostatin and ageing on satellite cell behaviour, the total number of satellite cells and their ability to become activated was quantified from 1 and 24 month old wild-type and myostatin-null mice.

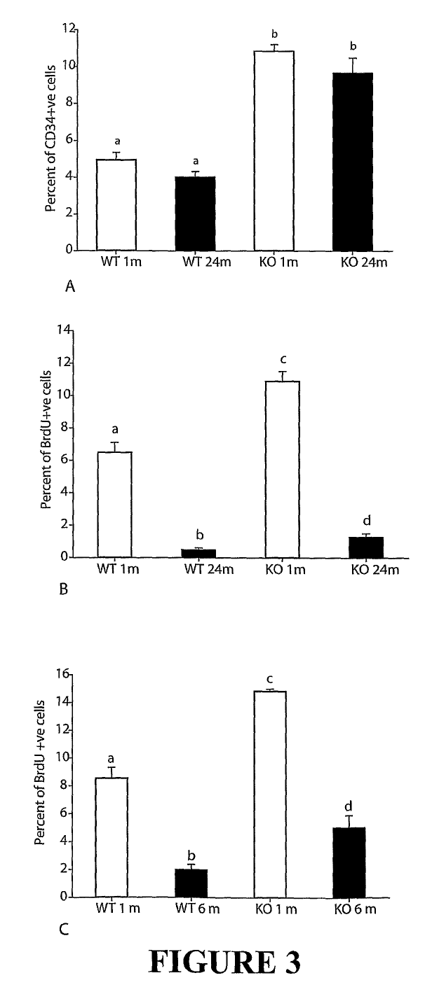

In order to analyse satellite cell numbers per unit fibre length, satellite cells attached to single fibres isolated from 1 and 24 month old wild-type and myostatin-null TA muscle were counted using the cell surface marker CD34 (FIG. 3A). Results indicated the average number of satellite cells per fibre 100 myonuclei increased significantly from 5 observed in 1 month old wild-type fibres to 11 in 1 month old myostatin-null fibres (FIG. 3A). Ageing appeared to have little effect on satellite cell number as no significant change in the satellite cell number was observed between 1 and 24 month old wild-type or myostatin-null fibres (FIG. 3A).

Since not only the number of satellite cells but also the activity of satellite cells is relevant to the ability of a muscle to regenerate, satellite cell activation was investigated using in vitro and in vivo BrdU labelling. In vitro BrdU labelled satellite cells attached to isolated fibres indicated the average percentage of activated satellite cells per fibre in 1 month old wild-type TA was 6.5% as opposed to 10% in 1 month old myostatin-null TA muscle (FIG. 3B). However, during ageing satellite cell activation was reduced in both the wild-type and myostatin-null 24 month old mice (FIG. 3B). It is noteworthy that at 24 months, there was still twice the number of activated satellite cells per fibre in myostatin-null muscle fibres as compared to wild-type fibres. Finally, the propensity of satellite cells to become activated was also measured using in vivo BrdU incorporation. FACS analysis of BrdU labelled satellite cells indicated similar trends to the in vitro labelled satellite cells. The percentage of activated satellite cells from 1 month old wild-type muscle was 8.5% as opposed to 14.8% in 1 month old myostatin-null muscle. With increasing age the percentage of activated satellite cells in both wild-type and myostatin-null six month old muscle dropped significantly to 2% and 5% respectively (FIG. 3C). It is noteworthy that in the myostatin null mice there is double the number of activatable satellite cells as compared to the controls.

### 350 Can Activate Satellite Cells

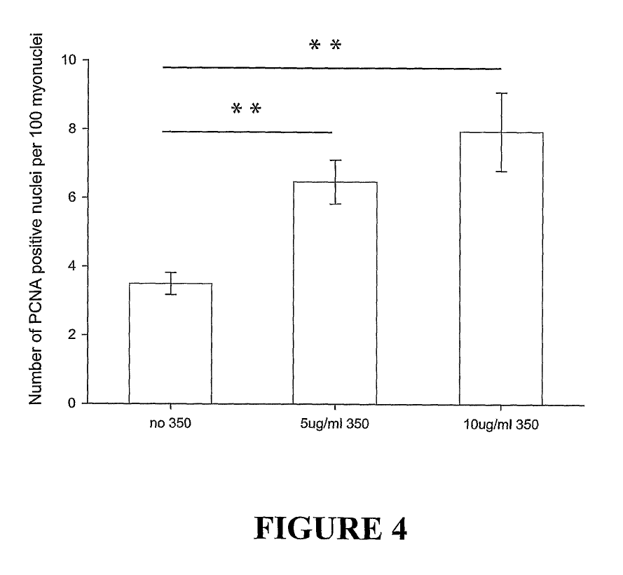

Because the physiological properties, including number per muscle fibre and degree of activation, of the satellite cells in the null mice may have been due to effects mediated during fetal development rather than due to lack of exposure to myostatin post-natally we tested the effect of a myostatin antagonist on satellite cell activation from wild type mice. When single muscle fibres from wild type mice containing satellite cells were incubated with increasing concentration of 350, increased number of satellite cell activation was observed. This result confirms that 350 is a potent activator of satellite cells in wild type muscle. It also indicates that the observation of increased satellite cell activation in myostatin null mice is likely to be due to continuing postnatal non-exposure to myostatin rather than from effects resulting from fetal non-exposure to myostatin. The finding that 350 can activate quiescent wild type satellite cells, in combination with the observation that myostatin null mice have increased levels of activated satellite cells during old age, indicates that administration of 350 or other myostatin antagonists can be expected to prevent the onset of conditions such as sarcopenia in older people. Furthermore it can be expected to reduce the severity of the condition in cases where the proportion of activated satellite cells has already commenced (FIG. 4)

### Example 2

### Myostatin Antagonists Increase Inflammatory Response and Chemotaxis of Satellite Cells

Sarcopenia is a form of muscle wasting associated with old age. With ageing, the reduction in muscle mass is accompanied by atrophy of muscle fibres. These events not only affect muscle fibres but also satellite cells, leading to reduced ability of muscle to regenerate. This is primarily due to loss of propensity of satellite cells to activate in response to injury and to the need for normal replenishment of muscle fibres. In addition, another major step of regeneration, inflammatory response to muscle injury, is also reduced in old age and is responsible for part of the symptoms of sarcopenia. Myostatin a potent negative regulator of myogenesis is shown to increase in circulation during ageing. Here we present data that confirms that increased myostatin levels are inhibitory to the activation of satellite cells and chemotaxis of inflammatory cells. We also provide evidence that a strong myostatin antagonist can reverse and rescue myostatin mediated inhibition of satellite cell activation and chemotaxis of inflammatory cells. These surprising findings indicate that myostatin inhibitors can act as a therapy for sarcopenia

### Materials and Methods

### Expression and Purification of 350

A cDNA corresponding to the 267-350 amino acids of bovine myostatin, hereafter referred to as 350 or 350 protein, was PCR amplified and cloned into pET16-B vector. Expression and purification of 350 protein was done according to the manufacturer's (Qiagen) protocol under native conditions.

### Notexin Model

Six to eight week old male C57BL/10 and Mstn^{-/-} mice (n=27 per group) were anaesthetized, using a mixture of 25% Hypnorm (Fentanyl citrate 0.315 mg/ml and Fluanisone 10 mg/ml) and 10% Hypnovel (Midazolam at 5 mg/ml) at 0.1 ml/10 g body weight. The tibialis anterior muscle of the right leg was injected intramuscular with 10 μl of 10 μg/ml Notexin, using a 100 μl syringe (SGE, Australia). Tibialis anterior muscles were removed from euthanized mice at day 0 (control), and days 1, 2, 3, 5, 7, 10, 14 or 28 (n=3 per day). The tibialis anterior muscles were mounted in Tissue Tec and frozen in isopentane chilled in liquid nitrogen. For trials of 350 on aged muscle, 1 year old wild type mice were injected with notexin as mentioned above into the left tibialis anterior (TA) muscle. Notexin injected mice were either injected subcutaneously with the myostatin antagonist, 350, at 6 μl per gram of body weight, or the equivalent amount of saline (control mice) on days 1, 3, 5, and 7. To assess the effect of 350 on muscle healing, mice were euthanized on days 1, 3, 7, 10 and 28 after injection of notexin and TA muscles were dissected out and processed for protein isolation or tissue sectioning. Frozen muscle samples were stored at −80° C. Seven μm transverse sections (n=3) were cut at 3 levels, 100 μm apart. The sections were then stained with hematoxylin and eosin or Van Geisen. Sections were then examined and photographed using an Olympus BX50 microscope (Olympus Optical Co., Germany) fitted with a DAGE-MTI DC-330 colour camera (DAGE-MTI Inc.).

### Immunohistochemistry

Frozen muscle sections (7 μm thick) were post fixed in 2% paraformaldehyde and then permeabilised in 0.3% (v/v) Triton X-100 in PBS and then blocked with 10% (v/v) normal goat serum-Tris buffered saline (NGS-TBS) for 1 hour at RT. The sections were incubated with antibodies diluted in 5% NGS-TBS overnight at 4° C. The antibodies used were mouse anti-MyoD, 1:25 dilution (554130; PharMingen) a specific marker for activated myoblasts (Cooper et al., 1999; Koishi et al., 1995); goat anti-Mac-1, 1:400 dilution (Integrin M-19; Santa Cruz) an antibody specific for infiltrating peripheral macrophages (Springer et al., 1979); mouse anti-vimentin antibody at 1:300 dilution a marker for fibroblasts. The sections were washed 3 times with PBS, then were incubated with either donkey anti-mouse Cy3 conjugate, 1:400 dilution (715-165-150; Jackson ImmunoResearch, West Grove, Pa. USA) or biotinylated donkey anti-sheep/goat IgG antibody 1:400 dilution RPN 1025; Amersham). Secondary antibody incubation was followed by incubation with streptavidin conjugated to fluorescein, 1:400 dilution (S-869; Molecular Probes) diluted in 5% NGS-TBS for 30 min at RT. Sections were rinsed with PBS 3 times, counter stained with DAPI and mounted with Dako® fluorescent mounting medium. Tibialis anterior muscle sections were examined by epi-fluorescent microscopy. Representative micrographs were taken on an Olympus BX50 microscope (Olympus Optical Co., Germany) fitted with a DAGE-MTI DC-330 colour camera (DAGE-MTI Inc., IN, USA). The average muscle area was measured using the Scion Imaging program (NIH with 5 random muscle sections used previously for immunohistochemistry from Mstn^{-/-} and wild type mice.

### Chemotaxis Assay

Primary myoblasts were cultured from the hind limb muscle of 4 to 6 week old mice, according to the published protocols (Allen et al., 1997; Partridge, 1997). Briefly, muscles were minced, and digested in 0.2% collagenase type 1A for 90 min. Cultures were enriched for myoblasts by pre-plating on uncoated plates for 3 hours. Myoblast cultures were maintained in growth media (GM) supplemented with 20% fetal calf serum (FCS), 10% HS and 1% CEE on 10% Matrigel coated plates, at 37° C./5% CO₂. The extent of culture purity was assessed by flow cytometry analysis of MyoD expression after 48 hours in culture. Cells were harvested using trypsin, suspended at a concentration of 10⁶ cells/200 μl and fixed overnight in 5 ml 70% ethanol at −20° C. Staining was performed for 30 min at room temperature using rabbit polyclonal anti-MyoD, 1:200 (Santa Cruz), followed by Alexa fluor 488 anti-rabbit conjugate, 1:500 (Molecular Probes). Analysis was carried out in duplicate with 10⁴ cell events collected in each assay. Debris was excluded by gating on forward and side scatter profiles. Cells were analyzed by FACScan (Becton Dickinson). Macrophages were isolated by a peritoneal lavage technique. Zymosan-activated mouse serum (ZAMS) was prepared according to the published protocol (Colditz and Movat, 1984). Chemotaxis experiments were performed in single blind-well Boyden-type chambers with 7 mm diameter wells (Neuro Probe, Md. USA). Standard polycarbonate filters with 8 μm holes (Neuro probe; holes=6% of surface area) were washed thoroughly, and for the myoblast assay, filters were treated with 1% Matrigel in DMEM for 30 min. Filters were then dried and placed between the top and bottom chambers.

For the chemotaxis assay of myoblasts, DMEM containing 5% chicken embryo extract (CEE) plus dialysis buffer was used as positive control. Recombinant myostatin (2.5 and 5 μg/ml myostatin) and 350 protein (at 5-times myostatin concentration, i.e., 12.5 μg/ml and 25 μg/ml) were added to positive control medium. Plain DMEM was used as negative control. On a 24-well plate, the bottom wells were filled with test or control media. Seventy-five thousand cells were added to the top wells. The plate was incubated for 7 h at 37° C., 5% CO₂. The top surface of the membranes was washed with pre-wet swabs to remove cells that did not migrate. The membrane was then fixed, stained in Gill's hematoxylin and wet mounted on slides. Migrated cells were counted on four representative fields per membrane and the average number plotted. For chemotaxis assay of macrophages, DMEM containing 33% Zymosan-activated mouse serum (ZAMS) plus dialysis buffer was used as positive control. Recombinant myostatin (5 μg/ml myostatin) and 350 protein (at 2 and 5-times myostatin concentration, i.e., 10 μg/ml and 25 μg/ml) were added to positive control medium or plain DMEM. On a 24-well plate, the bottom wells were filled with test or control media. Seventy-five thousand cells were added to the top wells containing polyethylene terephthalate (PET) 0.8 μm membranes. The plate was incubated for 4 h at 37° C., 5% CO₂. The top surface of the membranes was washed with pre-wet swabs to remove cells that did not migrate. The membrane was then fixed, stained in Gill's hematoxylin and wet mounted on slides. Migrated cells were counted on four representative fields per membrane and the average number plotted.

Primary fibroblasts were obtained from lamb skin explants. DMEM containing 10 pg/ml of recombinant TGF-β was used as positive control. Recombinant myostatin (5 μg/ml myostatin) was added to positive control media. On a 24-well plate, the bottom wells were filled with test or control media Eighty eight thousand cells were added to the top wells containing polyethylene terephthalate (PE) 0.8 μm membranes. The plate was incubated for 4 h at 37° C., 5% CO₂. The top surface of the membranes was washed with pre-wet swabs to remove cells that did not migrate. The membrane was then fixed, stained in Gill's hematoxylin and wet mounted on slides. Migrated cells were counted on four representative fields per membrane and the average number plotted.

### RT PCR for Gene Expression

Total RNA was isolated using Trizol (Invitrogen) according to the manufacturer's protocol. Reverse transcription reaction was performed using Superscript preamplification kit (Invitrogen). PCR was performed with 1111 of the reverse transcription reaction, at 94° C. for 30 s, 55° C. for 30 s, and 72° C. for 30 s. For each gene, number of cycles required for exponential amplification was determined using varying cycles. The amplicons were separated on an agarose gel and transferred to a nylon membrane. The PCR products were detected by Southern blot hybridization. Each data point was normalized by the abundance of glyceraldhyde-3-phosphate dehydrogenase (GAPDH) mRNA.

### Results

### Myostatin Influences the Chemotaxis of Myoblasts, Macrophages and Fibroblasts.

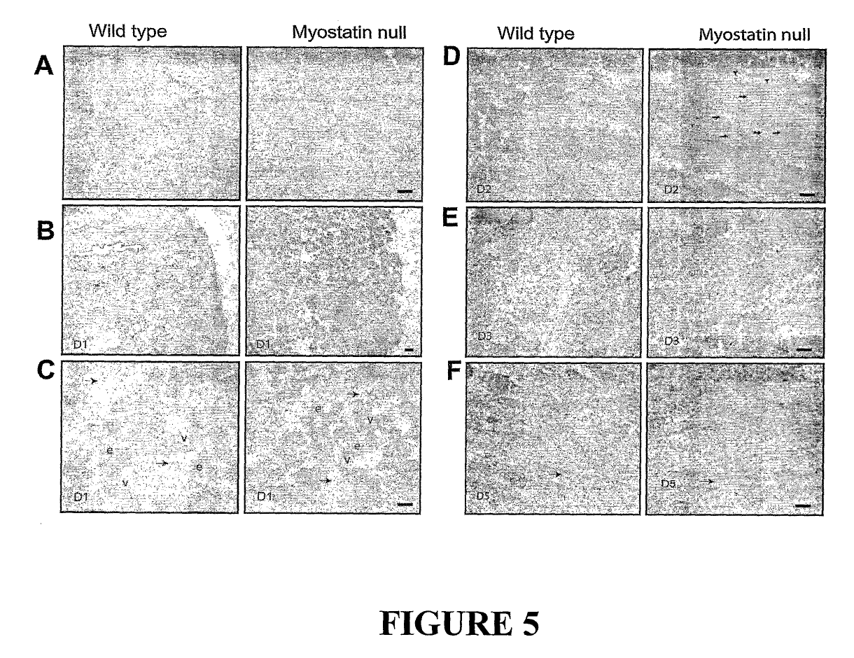

The inflammatory response is also involved in the regeneration cycle, for example in response to damaged or worn out muscle cells. The immune response is characterised by the presence of eosinophils, and myoblast migration was seen within 24 hours after notexin injection in both wild type and Mstn^{-/-} muscle (FIG. 5C). By day 2, the differences between wild type and Mstn^{-/-} responses in inflammatory response and satellite cell migration were pronounced with a marked increase in accretion of nuclei at the site of regeneration in Mstn^{-/-} muscle sections (FIG. 5D, arrows). Increased numbers of nuclei observed are due to increased numbers of macrophages and myoblasts. The highest density of nuclei was seen along the margins of the necrotic myofibres (FIG. 5D, arrowheads), particularly in Mstn^{-/-} sections. By day 3 regenerating wild type muscle sections also showed an increase in number of nuclei, although still far less than in comparable tissue collected from the Mstn^{-/-} mice (FIG. 5E). Accretion of mononuclear cells following notexin injection peaked at day 5 in both wild type and Mstn^{-/-} muscle sections (FIG. 5F). The major effect noted was an accelerated migration of macrophages and myoblasts to the regeneration site in Mstn^{-/-} muscle sections.

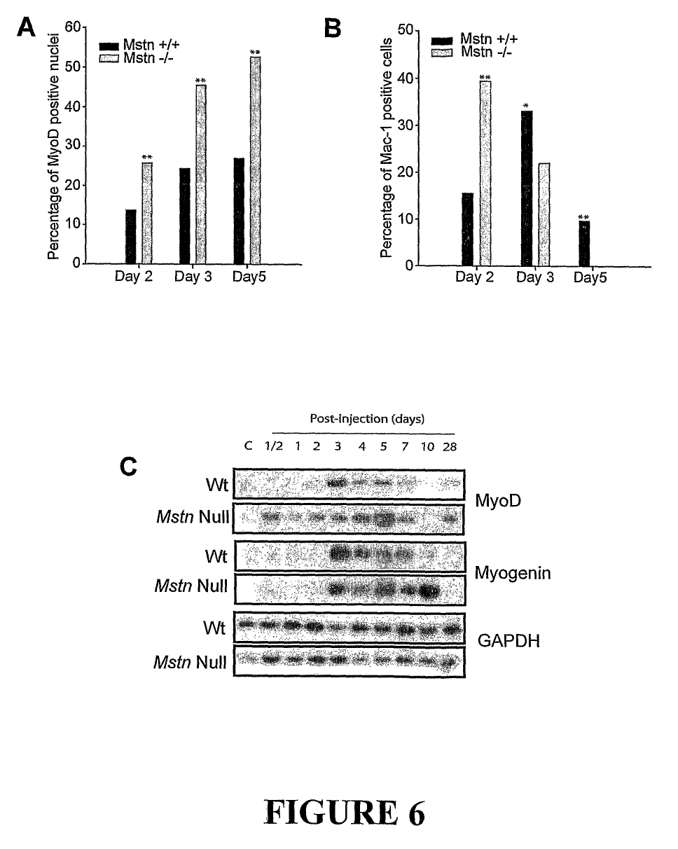

During muscle regeneration, inflammatory cells and satellite cells migrate to the site of regeneration (Watt et al., 1994). To determine if lack of myostatin enhances the migration of either activated satellite cells or inflammatory cells, the proportion of the inflammatory cells and myoblasts at the site of regeneration was quantified. Immunohistochemistry was used to detect MyoD, a specific marker for myoblasts (Beauchamp et al., 2000), and Mac-1, for infiltrating peripheral macrophages (Kawakami et al., 1995). Control untreated muscle sections were found to be negative for MyoD immunostaining. Muscle sections were stained with DAPI to count total number of nuclei. Quantification results demonstrate that in the Mstn^{-/-} regenerating muscle, twice the number of myogenic cells (MyoD positive) (FIG. 6A) and macrophages (Mac-1 positive) (FIG. 6B) are present at the site of regeneration at day 2 compared to the wild type sections. From day 2 through to day S post injection, Mstn^{-/-} muscle sections had more myoblasts than wild type muscle (FIG. 6A). Like the MyoD positive cells, the increased infiltration of macrophages to the site of regeneration was seen much earlier (on day 2) in the Mstn^{-/-} muscle in response to notexin injury (FIG. 6B). In addition, the inflammatory cell numbers decreased more rapidly in the Mstn^{-/-} muscle indicating that the whole process of inflammatory cell response was accelerated in Mstn^{-/-} mice (FIG. 6B).

Grounds et al (Grounds et al., 1992) demonstrated that MyoD and myogenin gene expression can be used as markers for the very early detection of migrating myoblasts during muscle regeneration. Hence the expression of MyoD and myogenin was determined in the regenerating tissue. Quantitative RT-PCR results confirm that the expression of the muscle regulatory factors myoD and myogenin, were expressed earlier in Mstn^{-/-} muscle as compared to wild type muscle. High levels of MyoD mRNA were detected within 12 hours after notexin injection in the Mstn^{-/-} muscle. In the wild type muscle however, MyoD expression was un-detectable until day 1 after notexin injection (FIG. 6C). Similarly, higher levels of mRNA for myogenin, was also detected very early within 12 hours after notexin injection in the regenerating Mstn^{-/-} muscle. However, in the wild type regenerating muscle, myogenin mRNA was not detected until 1 day after the muscle injury caused by notexin injection (FIG. 6C). Thus results from immunohistochemistry and gene expression analysis concur that there is increased and hastened migration of myogenic cells to the site of regeneration in Mstn^{-/-} muscle.

During old age a decrease in satellite cell activation and inflammatory response is seen in skeletal muscle. Based on the data presented here we propose that the increased levels of myostatin seen in ageing muscle contributes to the loss of propensity of satellite cells to be activated, both in response to injury and as needed prevent decrease of muscle bulk. In order to reverse these conditions seen in sarcopenia, we treated aged mice with myostatin antagonists.

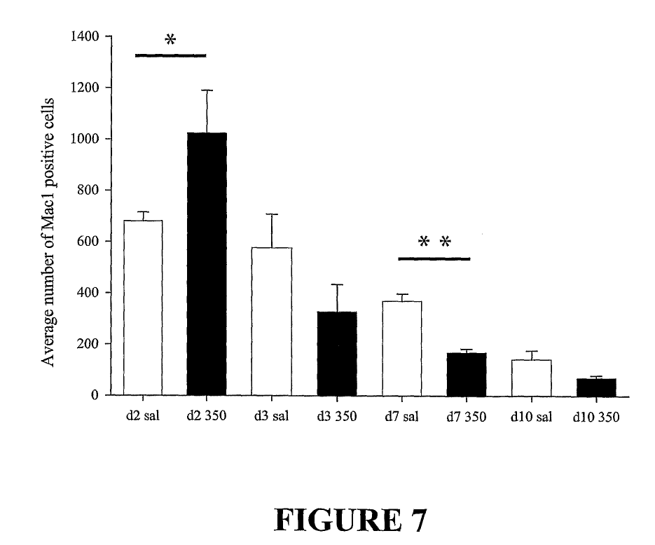

To demonstrate the beneficial effects of myostatin activity inhibition by 350 on enhanced inflammatory response, mice undergoing muscle regeneration after notexin injection were treated with 350 protein and inflammatory response was determined. A greater percentage of Mac1 positive macrophages were found in day 2 injured muscles which had been treated with 350 (FIG. 7). By day 3, the percentage had dropped in the 350 treated muscles below that of the saline treated day 3 muscles and continued to be lower in day 7 and 10 muscles. This result indicates an early or more profound recruitment of macrophages in the 350 treated muscles by day 2, followed by a decreased recruitment by day 7 and 10. These results show accelerated muscle inflammatory processes with the 350 treatment. The capacity for myostatin antagonists such as 350 to enhance the macrophage response by decreasing the inhibitory effects of myostatin indicates that administration of myostatin inhibitors or antagonists will have beneficial effects on people suffering sarcopenia, via a restoration of the inflammatory responses needed to maintain muscle integrity during ageing.

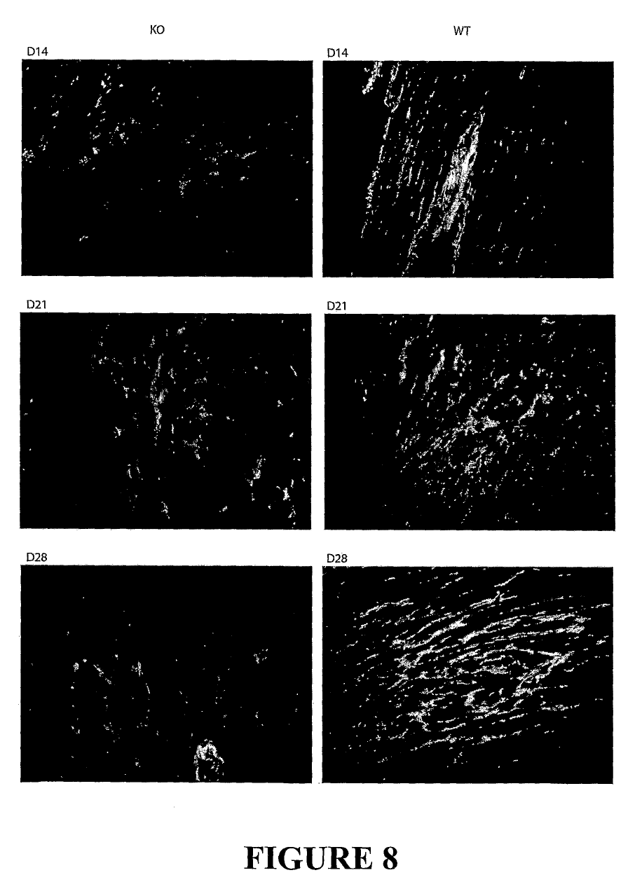

In addition to myoblasts, fibroblasts also migrate and populate the regeneration site. The effect of myostatin on the dynamics of fibroblast migration during muscle regeneration was investigated. As shown in FIG. 8 staining with vimentin antibody (a specific marker for fibroblasts) indicate that there is substantially less accretion of fibroblasts in the TA muscles in Mstn^{-/-} mice at the regeneration site as compared to wild type muscle. This result, in combination with data below on migration assays on fibroblasts, clearly demonstrates that myostatin acts as a chemoattractant for fibroblasts.

### Inhibition of Chemotaxis of Myoblasts and Macrophages by Myostatin and its Rescue by 350

It has been demonstrated that there is a significant fold increase in myostatin levels in muscle tissues injured by notexin after 24 hours (Kirk et al. 2000).

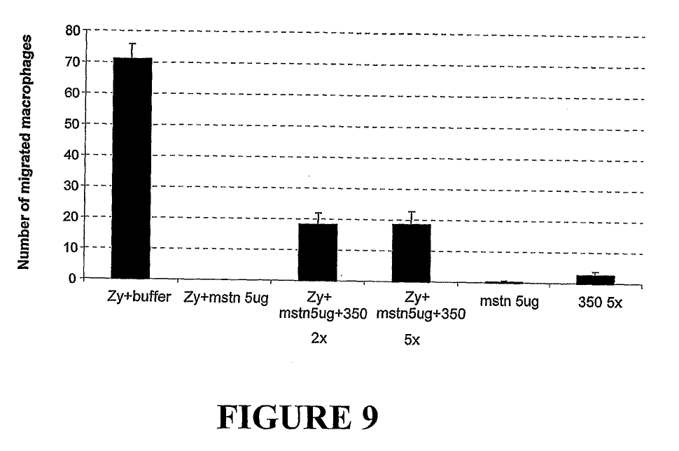

Results presented above indicate that Mstn^{-/-} muscle has an increased and accelerated infiltration of macrophages and migration of myoblasts to the area of regeneration. Since both cell types are known to be influenced by chemotactic factors to direct their movement (Bischoff, 1997; Jones, 2000) the effect of myostatin on the migratory ability of satellite cell derived myoblasts and macrophages was investigated. To test whether myostatin interferes with chemotactic signals, blind-well chemotaxis chambers were used. Isolated myoblasts or macrophages were assessed for their migratory ability through a filter towards a chemo-attractant (CEE for myoblasts, and ZAMS activated serum for macrophages). The isolated myoblasts were found to be 90% myogenic (MyoD positive) as assessed by flow cytometry. As shown in FIG. 9, addition of 5 μg/ml myostatin to ZAMS medium completely abolishes macrophage migration. When 350 protein is added to the medium containing μg/ml myostatin, a significant rescue of the chemo-inhibitory effect of myostatin on macrophages is observed (20-fold increase). This result confirms that administration of myostatin inhibitors such as 350 can accelerate muscle regeneration processes by decreasing the inhibition of macrophage migration by myostatin.

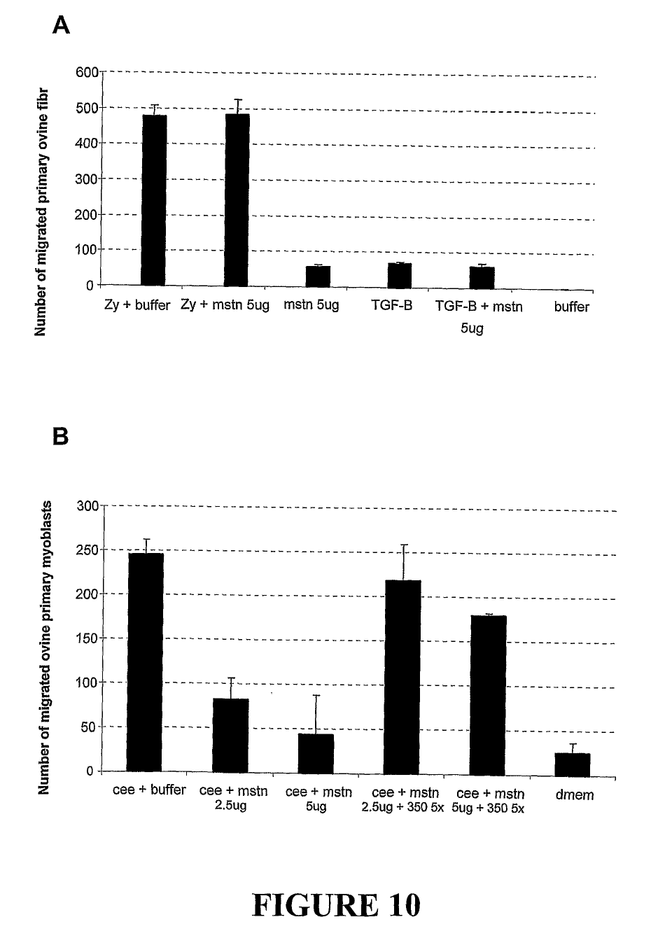

In addition to the effects on macrophage migration, here we also demonstrate that myostatin antagonists such as 350 can also decrease the negative effects of myostatin on the chemotactic movement of myoblasts. Addition of recombinant myostatin at 2.5 and 5 μg/ml to positive control medium leads to 66 and 82% inhibition of myoblast migration respectively. When 350 protein is added to the medium containing recombinant myostatin, the chemo-inhibitory effect of myostatin on myoblasts is rescued to levels similar to observed in the positive control thus demonstrating that myostatin antagonists such as 350 can effectively accelerate muscle regeneration by enhancing myoblast migration (FIG. 10B). The capacity for myostatin antagonists such as 350 to enhance myoblast migration by decreasing the inhibitory effects of myostatin indicates that administration of myostatin inhibitors will have beneficial effects on people suffering sarcopenia, via a restoration of the muscle regeneration responses needed to maintain muscle integrity during ageing.

### Myostatin Acts as a Chemo-Attractant for Fibroblasts

In contrast to the macrophages and myoblasts, myostatin acts as a chemotactic agent for the migration of fibroblasts. This is supported by the observation of reduced migration of fibroblasts to the regeneration site in the myostatin null muscle (FIG. 10A). To directly demonstrate the chemotactic effect of myostatin on the fibroblast, a migration assay was conducted in vitro using recombinant myostatin. As shown in FIG. 10A, addition of myostatin increases the chemotactic movement of fibroblasts as compared to the buffer control.

### Example 3

### Antagonizing Myostatin Results in Reduced Fibrosis and Enhanced Muscle Regeneration

### Methods

### Cut Injury Model

A 3 mm transversal incision was made on the left tibialis anterior (TA) of each mouse (wild type and myostatin null). On days 0, 3, 5, and 7 after injury the TAs of wild type were injected with either 350 protein at 2 μg/g body weight (total of 85 μg/mouse) or saline at the site of injury (into the TA muscle). The uninjured right TA was used as control. The injured and control muscle were collected at day 2, 4, 7, 10 and 21 after cutting and their weights determined. The extent of collagen deposition in regenerations and regenerated cut muscle tissue was also measured by Van Geisen staining.

### SE Microscope

The muscle samples were cleaned of fat and tendons and fixed in 10 ml of 0.1 M phosphate buffer (pH 7.4) containing 2.5% (v/v) glutaraldehyde for 48 hours with gentle rocking. The glutaraldehyde was washed off in PBS for 1 hour, before being transferred to 50 mls of 2 M NaOH, and incubated for 5 days at a constant 25° C. Samples were then washed in PBS, and transferred to 50 mls of sterile distilled water. Muscles were kept at a constant 25° C. for an additional 4 days. For the first 36 hours the water was changed every 12 hours, then every 24 hours there after. The muscles were then transferred to 1% tanic acid for 2 hours, and then washed in PBS 3 times. Muscle was treated with 1% OsO4 for 2 hours followed by dehydration by emersion 3 times for 15 min each into an ascending gradient of ethanol (50%-100%). Muscle samples were dried using carbon dioxide and coated with. gold. Specimens were examined and photographed using a scanning electron microscope (HITACHI 4100, Japan) with an accelerating voltage of 10 kV.

Collagen accumulation was assessed at day 21 in wild type versus null cut TAs using Van Geisen as described in Example 2.

### Results

### Lack of Myostatin Results in Enhanced Muscle Regeneration and Reduced Fibrosis

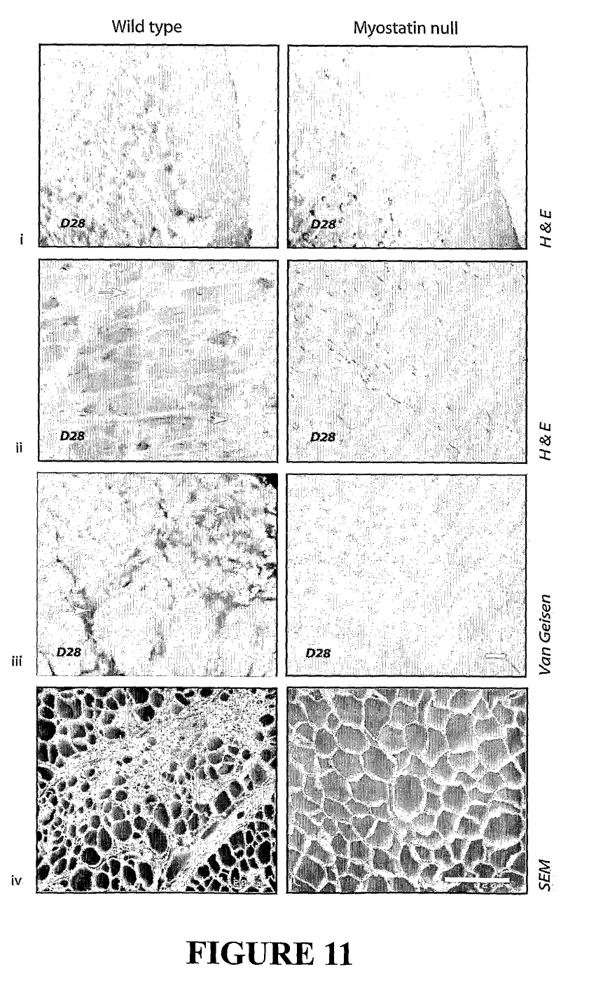

One of the hall marks of sarcopenia is the loss of muscular strength due to increased fibrosis. Repeated cycles of degeneration and regeneration of skeletal muscle during post-natal ageing results in accumulation of fibrotic tissue. To assess the role of myostatin in fibrosis, histology of both muscle genotypes were compared after notexin injection (see methods section in Example 2). At day 28, scar tissue was observed in hematoxylin and eosin stained sections from injured wild type muscle, while very little was seen in the Mstn^{-/-} muscle sections (FIG. 11A). The presence of connective tissue was further confirmed by Van Geisen's stain (FIG. 11A). Wild type muscle sections at day 28 had larger areas of collagen, therefore more scar tissue was seen in the cut wild type tissue as compared to the Mstn^{-/-} muscle. To further confirm this result, regenerated muscle was analyzed using scanning electron microscopy. Scanning electron micrographs of day 0 (control) and day 24 regenerated muscle, showed the connective tissue framework surrounding the spaces once occupied by the myofibres (FIG. 11A). Neither wild type nor Mstn^{-/-} muscle had thickened connective tissue around the fibre cavity in the control (not injured) samples. However, by day 24 of muscle regeneration dense bundles of connective tissue were observed in the wild type muscle (FIG. 11A), but not in the Mstn^{-/-} muscle. Similarly, in a cut muscle model comparing myostatin null versus wild type mice the degree of collagen accumulation at the regenerated muscle site at day 28 was significantly reduced in myostatin null mice (data not presented). These results confirm that lack of myostatin leads to reduced scar tissue after muscle regeneration. This can be expected to aid in reduction of scar tissue in ageing muscle and thus decrease the symptoms of sarcopenia.

### 350 Treatment Enhances Muscle Regeneration and Reduces Fibrosis

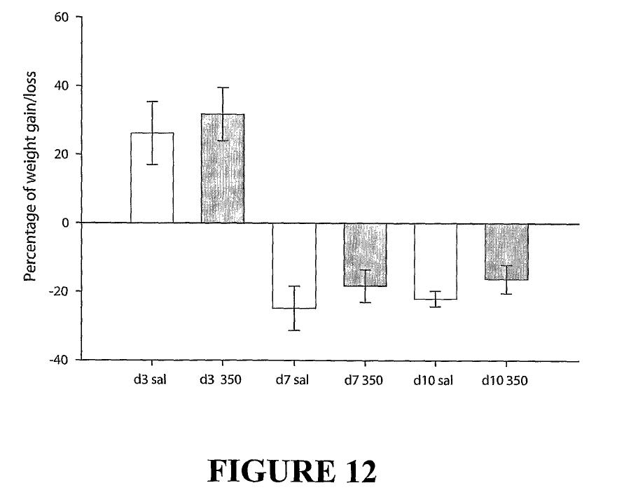

In order to study the efficacy of myostatin antagonists such as 350 in enhancing muscle regeneration, 1 year old wild type mice (C57 Black) were injured with notexin and injected with 350 (see methods in example 2). After notexin injury, typically the muscle weight initially increases due to the resulting oedema, followed by a decrease due to necrosis of the damaged muscle fibres which are cleared from the site of injury. After this time, the muscle weight begins to increase again due to growth of new fibres. Results from the trial show that 350 treated muscles do not lose as much weight as control saline injected muscle do (FIG. 12) at day 7 and 10. This is probably due to faster repair of damaged muscle. Molecular data presented (FIG. 7) does indeed support the hypothesis that in 350 treated mice, the damaged muscle regenerated much faster due to a combination of accelerated and enhanced macrophage migration and the other accelerated muscle regeneration processes discussed earlier that are associated with the use of myostatin antagonists to treat sarcopenia Histological analysis confirmed variations between the saline and 350 treated muscles.

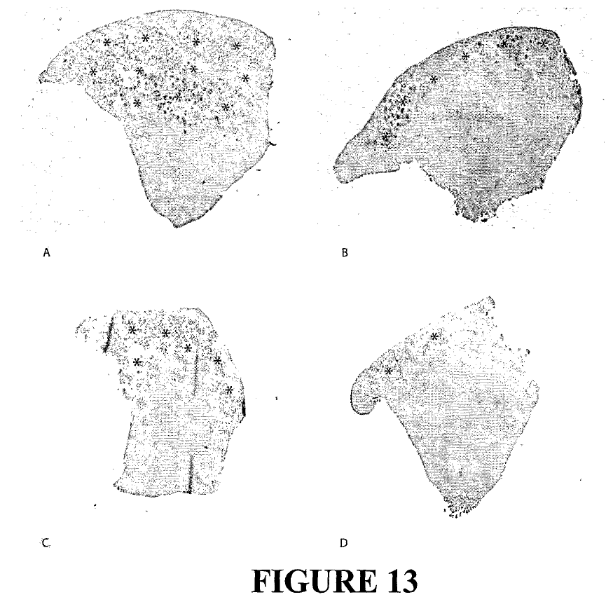

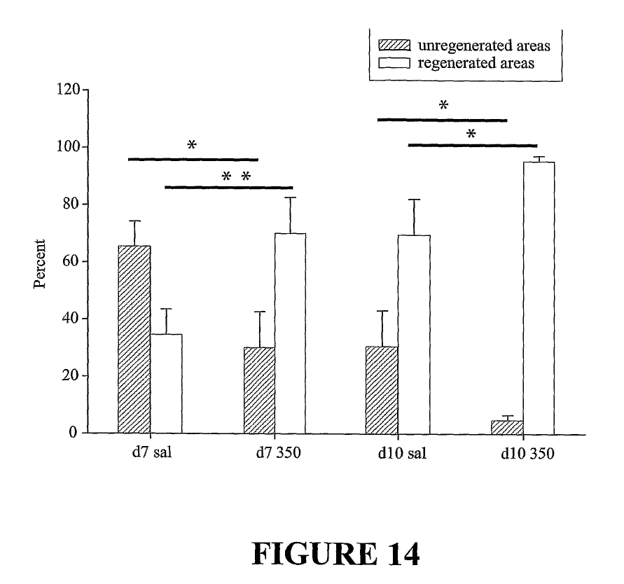

Haematoxylin and eosin staining indicated earlier nascent muscle fibre formation and an associated earlier reduction in necrotic areas in the muscles treated with 350 compared to saline treated muscles (FIG. 13). This result confirms accelerated and enhanced muscle regeneration in 350 treated mice. The histological data shown in FIG. 9 was analysed to quantify both regenerated and un-regenerated areas of the whole muscle cross-sectional view area. The muscle sections were consistently taken from the mid belly region of each muscle. The analysis shown in FIG. 14, indicates that at day 7 in the saline treated control mice there is increased un-regenerated area as compared to 350 treated mice. As a result there is a relatively less regenerated muscle in controls as compared to 350 treated mice at day 7. The same effect is seen at day 10 also. These results confirm that while there is a decrease in the un-regenerated area, there is increase in the regenerated area in 350 treated muscle as compared to saline treated controls.

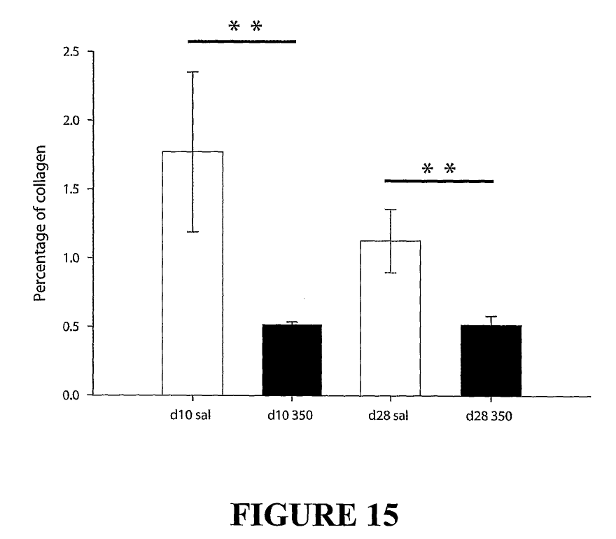

In addition, Van Geisen staining, which detects collagen, showed reduced levels of collagen deposition in 350 treated muscles compared to saline treated muscles, at 10 and 28 days after the administration of notexin indicating that the 350 treatment reduced fibrosis during the muscle regeneration process (FIG. 15). This result demonstrates that myostatin antagonists such as 350 reduce scar tissue (fibrosis) formation during muscle regeneration. This shows that administration of myostatin antagonists such as 350 can be expected to aid in reduction of scar tissue in ageing muscle and thus decrease the symptoms of sarcopenia.

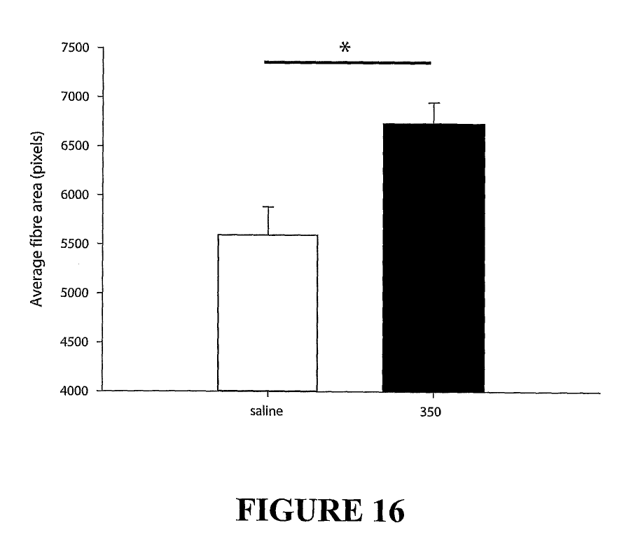

Using the Van Geisen stained images, randomly selected regenerated fibre areas were measured to assess fibre size at 28 days after the administration of notexin (FIG. 16). Results from this analysis indicated that the regenerated fibres from 350 treated muscles were significantly larger than the saline treated muscles. The increased repaired muscle fibre size confirms the induction of hypertrophy in muscle cells due to inhibition of myostatin function by 350.

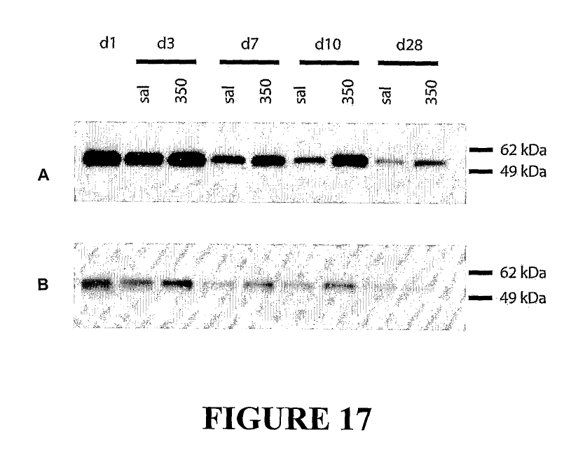

To further confirm that increased muscle regeneration in 350 treated mice is due in part to increased activation of satellite cells we performed molecular analysis for the expression of Pax7 and MyoD proteins. Pax7 protein is a maker for satellite cells and expression of MyoD indicate the activation of satellite cells. Protein analysis confirmed increased levels of satellite cell and activation (FIG. 17). Pax7 levels (FIG. 17A) were higher with 350 treatment at days 3, 7, 10, and 28, indicating an increase in satellite cell activation compared to saline treated muscles. In addition, in the 350 treated muscles, the level of Pax7 increased between day 7 and 10 in contrast to a decrease observed in the saline treated muscle. This would indicate an increase of satellite cell activation around day 10 in the 350 treated muscles. MyoD levels (FIG. 17B) were also higher with 350 treatment at days 3, 7, and 10 showing increased myogenesis compared to the saline treated muscles. Taken together, higher Pax7 and MyoD levels in 350 treated tissues support the observation that activation of satellite cells, and therefore subsequent myogenesis is increased. This result confirms that treatment with 350 accelerates and enhances muscle regeneration and will decrease the symptoms of sarcopenia.

### Local Application of 350 Induced Enhanced Muscle Regeneration

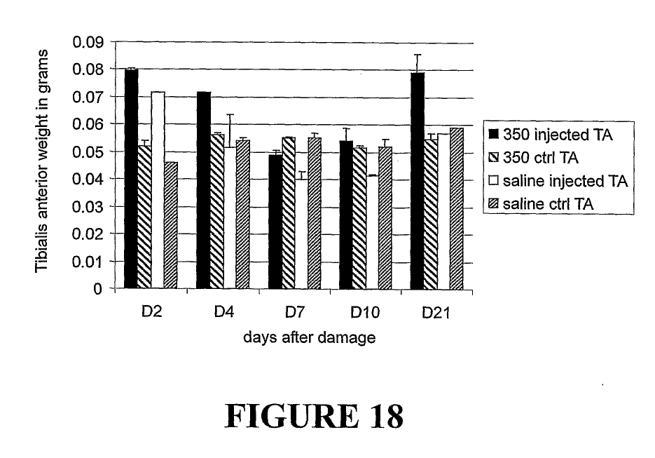

To assess the effectiveness of direct application of 350 at the muscle regeneration site in enhancing muscle regeneration, 350 protein was applied to the TA muscle that was regenerating after damage was inflicted by cutting as described above. The uninjured right TA was used as control. The injured and control muscles were collected at day 2, 4, 7, 10 and 21 after damaging and their weight determined. An initial increase in muscle weight due to inflammatory infiltration is observed in both 350 and saline injected TAs at day 2 and 4 after cutting (FIG. 18). At day 7 to 10 after damaging the muscles recover their normal weight in both 350 and saline injected TAs. However, at day 21 after damaging, the 350 injected TAs display a significant increase in muscle size as reflected in muscle weight compared to saline treated muscles.

### Discussion

Sarcopenia is an age related loss of muscle mass and strength. The decreased muscle mass is caused in part by reduction in satellite cell activation and consequently ability of muscle to regenerate after damage and to maintain normal processes of muscle replenishment over time during ageing. The slower rate of inflammatory response and the reduced number of myoblasts are the primary contributing factors for reduced muscle regeneration during old age. Recently the levels of a potent negative regulator of muscle growth, myostatin, has been shown to be higher in older men and woman. Data documented here clearly demonstrates that myostatin inhibits satellite cell activation and inflammatory response. Thus we propose that myostatin is involved in the progression of sarcopenia Data presented here also demonstrates that either lack of myostatin or inhibition of myostatin activity by 350 results in increased activation of satellite cells and inflammatory response during muscle wasting. Since 350 is able to profoundly activate satellite cells, administration of 350 would result in activation of inflammatory response and the regeneration and replenishment of muscles tissues during ageing via processes driven by satellite cell activation. This will further lead to enhanced chemotaxis of both macrophages and myoblasts to the regenerating area. Since lack of myostatin also results in increased proliferation of myoblasts, this will further lead to increased myogenesis, successful repair of muscle damage and increased replenishment of muscles during ageing. Indeed in vivo trial data presented here clearly document that 350 administration can enhance muscle regeneration, thus confirming that 350 and other myostatin antagonists will be a valuable therapeutic option for sarcopenia treatment.

Due to repeated cycle of muscle damage and regeneration, there is increased fibrosis of muscle leading to reduced muscle strength During muscle regeneration fibrosis is contributed by the infiltrating fibroblasts. We have clearly shown here that myostatin acts as a chemotacting agent for fibroblasts migration. On the contrary, lack of myostatin results in the reduction of fibroblasts. When 350 was administered during muscle regeneration, we observed a reduction in fibrosis. Hence it is proposed that 350 administration during sarcopenia will also help alleviate fibrosis in muscles that occurs during ageing and will increase muscle strength in ageing muscles.

### CONCLUSION

Myostatin antagonists are able to successfully improve muscle mass by increasing muscle regeneration and reducing fibrosis in aged muscle. Therefore, myostatin antagonists will provide a valuable treatment option for the treatment and/or prevention of sarcopenia

### REFERENCES

Allen, R. E., Temm-Grove, C. J., Sheehan, S. M. and Rice, G. (1997). Skeletal muscle satellite cell cultures. Methods Cel Biol 52, 155-76.
Beauchamp, J. R., Heslop, L., Yu, D. S., Tajbakhsh, S., Kelly, R. G., Wernig, A., Buckingham, M. B., Partridge, T. A. and Zammit, P. S. (2000). Expression of CD34 and Myf5 defines the majority of quiescent adult skeletal muscle satellite cells. J Cel Biol 151, 1221-34.
Bischoff, R. (1994). Myology, vol. 1 (eds A. G. Engel and C. Franzi-Armstrong), pp. 97-118: McGraw-Hill Professional.
Carlson, C. J., Booth, F. W. and Gordon, S. E. (1999). Skeletal muscle myostatin mRNA expression is fiber-type specific and increases during hindlimb unloading. Am J Physiol 277, R601-6.
Colditz, I. G. and Movat, H. Z. (1984). Kinetics of neutrophil accumulation in acute inflammatory lesions induced by chemotaxins and chemotaxinigens. J Immunol 133, 2169-73.
Conboy, I. M. and Rando, T. A. (2002). The regulation of notch signaling controls satellite cell activation and cell fate determination in postnatal myogenesis. Dev Cel 3, 397-409.
Cooper, R. N., Tajbakhsh, S., Mouly, V., Cossu, G., Buckingham, M. and Butler-Browne, G. S. (1999). In vivo satellite cell activation via Myf5 and MyoD in regenerating mouse skeletal muscle. J Cel Sci 112 (Pt 17), 2895-901.
Floss, T., Arnold, H. H. and Braun, T. (1997). A role for FGF-6 in skeletal muscle regeneration. Genes Dev 11, 2040-51.
Gonzalez-Cadavid, N. F., Taylor, W. B., Yarasheski, K., Sinha-Hikim, I., Ma, K., Ezzat, S., Shen, R., Lalani, R., Asa, S., Mamita, M. et al. (1998). Organization of the human myostatin gene and expression in healthy men and HIV-infected men with muscle wasting. Proc Natl Acad Sci USA 95, 1493843.
Greenlund, L J S, Nair K S (2003) Sarcopenia—consequences, mechanisms and potential therapies. Mechanisms & Ageing and Development. 124: 287-299.
Grobet L, Martin L J, Poncelet D, et al. (1997) A deletion in the bovine myostatin gene causes the double-muscled phenotype in cattle. Nat Genet. 17:71-74.
Grounds, M. D., Garrett, K L. and Beilharz, M. W. (1992). The transcription of MyoD1 and myogenin genes in thymic cells in vivo. Exp Cel Res 198, 357-61.
Grounds, M. D. and Yablonka-Reuveni, Z. (1993). Molecular and cell biology of skeletal muscle regeneration. Mol Cel Biol Hum Dis Ser 3, 210-56.
Jones, G. E. (2000). Cellular signaling in macrophage migration and chemotaxis. J Leukoc Biol 68, 593-602.
Kambadur, R., Sharma, M., Smith, T. P. and Bass, J. J. (1997) Mutations in myostatin (GDF-8) in double muscled Belgian Blue and Piedmontese Cattle. Genome Res 7: 910-916.
Kawakami, K., Teruya, K., Tohyama, M., Kudeken, N., Yonamine, Y. and Saito, A. (1995). Mac1 discriminates unusual CD4-CD8-double-negative T cells bearing alpha beta antigen receptor from conventional ones with either CD4 or CD8 in murine lung. Immunol Lett 46, 143-52.
Kirk S., Oldham J., Kambadue R., Sharma., Dobbie P. and Bass J. (2000). Myostatin regulation during skeletal muscle regeneration. Journal of Cellular Physiology 184(3): 356-63.
Langley, B., Thomas, M., Bishop, A., Sharma, M., Gilmour, S, and Kambadur, R. (2002). Myostatin Inhibits Myoblast Differentiation by Down-regulating MyoD Expression. J Biol Chem 277, 49831-40.
Lee S J and McPherron A C (2001). Regulation of myostatin activity and muscle Growth. Procedings of National Academy of Science 98, 9306-9311
Lescaudron, L., Creuzet, S. B., Li, Z., Paulin, D. and Fontaine-Perus, J. (1997). Desmin-lacZ transgene expression and regeneration within skeletal muscle transplants. J Muscle Res Cel Motil 18, 631-41.
Lescaudron, L., Li, Z., Paulin, D. and Fontaine-Perus, J. (1993). Desmin-lacZ transgene, a marker of regenerating skeletal muscle. Neuromuscul Disord 3, 419-22.
McCroskery, S., Thomas, M., Maxwell, L., Sharma, M. and Kambadur, R. (2003). Myostatin negatively regulates satellite cell activation and self-renewal. The Journal of Cel Biology. 162.
McPherron, A. C., Lawler, A. M. and Lee, S. J. (1997). Regulation of skeletal muscle mass in mice by a new TGF-beta superfamily member. Nature 387, 83-90.
McPhexron A C, Lee S J. (1997b) Double muscling in cattle due to mutations in the myostatin gene. Proc Natl Acad Sci USA 94:12457-12461.
Merly, F., Lescaudron, L., Rouaud, T., Crossin, F. and Gardahaut, M. F. (1999). Macrophages enhance muscle satellite cell proliferation and delay their differentiation. Muscle Nerve 22, 724-32.
Nakamura K., Murata C., Ito M., Iwamori T., Nishimura S., Hisamatsu K., Sonoki S., Nakayama A., Suyama E., Kawasaki H., Taira k., Nishino K. and Tachi C. (2005) Design of hammerheas ribozymes that cleave murin sry mRNA in vitro and in vivo. Journal of Reproductive Development 17: epublished. Partridge, T. A. (1997). Tissue culture of skeletal muscle. Methods Mol Biol 75, 131-44.
Phillips, G. D., Lu, D. Y., Mitashov, V. I. and Carlson, B. M. (1987). Survival of myogenic cells in freely grafted rat rectus femoris and extensor digitorum longus muscles. Am J Anat 180, 365-72.
Raybum E., Wang w., Zhang R and Wang H. (2005) Antisense approaches in drug discovery and development. Progress in Drug Research 63: 227-74.
Ren Y., Gong W., Xu Q., Zheng X., Lin D., Wang Y. and Li T. (2006) siRecords: an extensive database of mammalian siRNAs with efficacy ratings. Bioinformatic 29:epublished
Rosenblatt, J. D., Lunt, A. I., Parry, D. J. and Partridge, T. A. (1995). Culturing satellite cells from living single muscle fiber explants. In Vitro Cel Dev Biol Anim 31, 773-9.
Springer, T., Galfre, G., Secher, D. S, and Milstein, C. (1979). Mac-1: a macrophage differentiation antigen identified by monoclonal antibody. Eur I Immunol 9, 3016.
Thomas, M., Langley, B., Berry, C., Sharma, M., Kirk S., Bass, J. and Kambadur, R. (2000). Myostatin, a negative regulator of muscle growth, functions by inhibiting myoblast proliferation. J Biol Chem 275, 4023543.
Watt, D. J., Morgan, J. E., Clifford, M. A. and Patridge, T. A. (1987). The movement of muscle precursor cells between adjacent regenerating muscles in the mouse. Anat Embryol (Berl) 175, 527-36
Wehling, M., Cai, B. and Tidball, J. G. (2000). Modulation of myostatin expression during modified muscle use. Faseb J 14, 103-10.
Yablonka-Reuveni N and Nameroft M (1987) Skeletal muscle cell populations. Separation and partial characterization of fibroblast-like cells from embryonic tissue using density centrifugation. Histochemistry. 87 27-38.
Yang L., Scott P. O., Dodd C., Medina A., Jiao H., Shankowsky H. A., Ghahary A. and Tredget E. E. (2005). Identification of fibrocytes in postburn hypertrophic scar. Wound Repair and Regeneration 13(4): 398404.
Zimmers, T. A., Davies, M. V., Koniaris, L. O., Haynes, P., Esquela, A. F., Tomkinson, K. N., McPherron, A. C., Wolfman, N. M. and Lee, S. J. (2002). Induction of cachexia in mice by systemically administered myostatin. Science 296, 148

### CITED PATENTS

U.S. Pat. No. 6,096,506, U.S. Pat. No. 6,369,201, U.S. Pat. No. 6,468,535, US 2004/0181033, US 2002/0181033, WO 02/085306, WO 01/53350, WO 01/05820, WO 00/43781, PCT/NZ2005/000250, PCT/NZ2004/000308

All references cited herein are hereby incorporated into the present specification by reference.

### INDUSTRIAL APPLICATION

The present invention provides a method for treating sarcopenia by administering one or more myostatin antagonists to a patient in need thereof. The method provides for improved muscle mass in aged muscle, as well as a reduction in collagen formation in regenerating muscle tissue, thereby improving overall functionality of the regenerated muscle tissue.

1. A method of treating sarcopenia comprising the step of administering an effective amount of at least one myostatin antagonist to a human or non-human patient in need thereof.

2. A method as claimed in claim 1, wherein the at least one myostatin antagonist is selected from the group consisting of:
an anti-myostatin antibody;
a myostatin peptide immunogen, myostatin multimer or myostatin immuno-conjugate capable of eliciting an immune response and blocking myostatin activity;
a protein inhibitor of myostatin selected from a truncated Activin type II receptor, a myostatin pro-domain and follistatin, or a functional fragment of said protein inhibitor;
a myostatin inhibitor released into culture from cells overexpressing myostatin;
a dominant negative of myostatin selected from the Piedmontese allele and mature myostatin peptides having a C-terminal truncation at a position at or between amino acid positions 335 to 375;
a small peptide comprising the amino acid sequence WMCPP and which is capable of binding to and inhibiting myostatin;
a splice variant of myostatin;
a regulator of the myostatin pathway; and
an antisense polynucleotide, RNAi, siRNA or an anti-myostatin ribozyme capable of inhibiting myostatin activity by inhibiting myostatin gene expression.

3. A method as claimed in claim 2, wherein the at least one myostatin antagonist is a dominant negative of myostatin selected from the Piedmontese allele and mature myostatin peptides having a C-terminal truncation at a position at of between amino acid positions 335 to 375.

4. A method as claimed in claim 3, wherein the at least one myostatin antagonist is a mature myostatin peptide having a C-terminal truncation at amino acid position 335 or 350.

5. A method as claimed in claim 2, where the at least one myostatin antagonist is a splice variant of myostatin selected from a polypeptide of SEQ ID NOS: 8-14, or a functional fragment or variant thereof, or a sequence having 95%, 90%, 85%, 80%, 75% or 70% sequence identify thereto.

6. A method as claimed in claim 2, wherein the at least one myostatin antagonist is a regulator of the myostatin pathway comprising the “mighty” peptide of SEQ ID NO: 16 or SEQ ID NO: 18, or a functional fragment or variant thereof, or a sequence having at least 95%, 90%, 85%, 80%, 75%, or 70% sequence identify thereto.

7. A method as claimed in claim 1, for increasing the activation of satellite cells, and migration of myoblasts and macrophages in a regenerating muscle.

8. A method as claimed in claim 1, wherein one or more additional growth promoting compounds selected from the group consisting of HGF, FGF, IGF, MGF and growth hormone are co-administered either separately, sequentially or simultaneously with the at least one myostatin antagonist to further improve muscle regeneration.

9. A method as claimed in claim 1, wherein the at least one myostatin antagonists is formulated for local or systemic administration.

10. A method as claimed in claim 9, wherein the at least one myostatin antagonist is formulated for oral, intravenous, cutaneous, subcutaneous, intradermal, nasal, pulmonary, intramuscular or intraperitional administration.

11. A use of at least one myostatin antagonist in the manufacture of a medicament for treating sacropenia in a human or non-human patient in need thereof.

12. A use as claimed in claim 11, wherein the at least one myostatin antagonist is selected from the group consisting of:
an anti-myostatin antibody;
a myostatin peptide immunogen, myostatin multimer or myostatin immuno-conjugate capable of eliciting an immune response and blocking myostatin activity;
a protein inhibitor of myostatin selected from a truncated Activin type II receptor, a myostatin pro-domain and follistatin, or a functional fragment of said protein inhibitor;
a myostatin inhibitor released into culture from cells overexpressing myostatin;
a dominant negative of myostatin selected from the Piedmontese allele and mature myostatin peptides having a C-terminal truncation at a position at or between amino acid positions 335 to 375;
a small peptide comprising the amino acid sequence WMCPP and which is capable of binding to and inhibiting myostatin;
a splice variant of myostatin;
a regulator of the myostatin pathway; and
an antisense polynucleotide, RNAi, siRNA or an anti-myostatin ribozyme capable of inhibiting myostatin activity by inhibiting myostatin gene expression.

13. A use as claimed in claim 12, wherein the at least one myostatin antagonist is a dominant negative of myostatin selected from the Piedmontese allele and mature myostatin peptides having a C-terminal truncation at a position at of between amino acid positions 335 to 375.

14. A use as claimed in claim 13, wherein the at least one myostatin antagonist is a mature myostatin peptide having a C-terminal truncation at amino acid position 335 or 350.

15. A use as claimed in claim 12, where the at least one myostatin antagonist is a splice variant of myostatin selected from a polypeptide of SEQ ID NOS: 8-14, or a functional fragment or variant thereof, or a sequence having 95%, 90%, 85%, 80%, 75% or 70% sequence identify thereto.

16. A use as claimed in claim 12, wherein the at least one myostatin antagonist is a regulator of the myostatin pathway comprising the “mighty” peptide of SEQ ID NO: 16 or SEQ ID NO: 18, or a functional fragment or variant thereof, or a sequence having at least 95%, 90%, 85%, 80%, 75%, or 70% sequence identify thereto.

17. A use as claimed in claim 11, wherein the medicament further comprises one or more additional muscle growth promoting compounds selected from the group consisting of HGF, FGF, IGF, MGF and growth hormone, and wherein the medicament is formulated for separate, sequential or simultaneous administration of the at least one myostatin antagonist and additional compound.

18. A use as claimed in claim 11, wherein the medicament is formulated for local or systemic administration.

19. A use as claimed in claim 18, wherein the medicament is formulated for oral, intravenous, cutaneous, subcutaneous, intradermal, nasal, pulmonary, intramuscular or intraperitional administration.

20. A pharmaceutical compound comprising at least one myostatin antagonist and a pharmaceutically acceptable carrier, when used in a method of treating sarcopenia in a human or non-human patient in need thereof.

21. A pharmaceutical compound as claimed in claim 20, wherein the at least one myostatin antagonist is selected from the group consisting of:
an anti-myostatin antibody;
a myostatin peptide immunogen, myostatin multimer or myostatin immuno-conjugate capable of eliciting an immune response and blocking myostatin activity;
a protein inhibitor of myostatin selected from a truncated Activin type II receptor, a myostatin pro-domain and follistatin, or a functional fragment of said protein inhibitor;
a myostatin inhibitor released into culture from cells overexpressing myostatin;
a dominant negative of myostatin selected from the Piedmontese allele and mature myostatin peptides having a C-terminal truncation at a position at or between amino acid positions 335 to 375;
a small peptide comprising the amino acid sequence WMCPP and which is capable of binding to and inhibiting myostatin;
a splice variant of myostatin;
a regulator of the myostatin pathway; and
an antisense polynucleotide, RNAi, siRNA or an anti-myostatin ribozyme capable of inhibiting myostatin activity by inhibiting myostatin gene expression.

22. A pharmaceutical compound as claimed in claim 21, wherein the at least one myostatin antagonist is a dominant negative of myostatin selected from the Piedmontese allele and mature myostatin peptides having a C-terminal truncation at a position at of between amino acid positions 335 to 375.

23. A pharmaceutical compound as claimed in claim 22, wherein the at least one myostatin antagonist is a mature myostatin peptide having a C-terminal truncation at amino acid position 335 or 350.

24. A pharmaceutical compound as claimed in claim 21, where the at least one myostatin antagonist is a splice variant of myostatin selected from a polypeptide of SEQ ID NOS: 8-14, or a functional fragment or variant thereof, or a sequence having 95%, 90%, 85%, 80%, 75% or 70% sequence identify thereto.

25. A pharmaceutical compound as claimed in claim 21, wherein the at least one myostatin antagonist is a regulator of the myostatin pathway comprising the “mighty” peptide of SEQ ID NO: 16 or SEQ ID NO: 18, or a functional fragment or variant thereof, or a sequence having at least 95%, 90%, 85%, 80%, 75%, or 70% sequence identify thereto.

26. A pharmaceutical compound as claimed in claim 20, further comprising one or more additional muscle growth promoting compounds selected from the group consisting of HGF, FGF, IGF, MGF and growth hormone, wherein the composition is formulated for separate, sequential or simultaneous administration with the at least one myostatin antagonist.

27. A pharmaceutical composition as claimed in claim 20, formulated for local or systemic administration.

28. A pharmaceutical compound as claimed in claim 27, formulated for oral, intravenous, cutaneous, subcutaneous, intradermal, nasal, pulmonary, intramuscular or intraperitional administration.

29. At least one myostatin antagonist when used in a method of treating sacropenia in a human or non-human patient in need thereof.

30. At least one myostatin antagonist as claimed in claim 29, is selected from the group consisting of:
an anti-myostatin antibody;
a myostatin peptide immunogen, myostatin multimer or myostatin immuno-conjugate capable of eliciting an immune response and blocking myostatin activity;
a protein inhibitor of myostatin selected from a truncated Activin type II receptor, a myostatin pro-domain and follistatin, or a functional fragment of said protein inhibitor;
a myostatin inhibitor released into culture from cells overexpressing myostatin;
a dominant negative of myostatin selected from the Piedmontese allele and mature myostatin peptides having a C-terminal truncation at a position at or between amino acid positions 335 to 375;
a small peptide comprising the amino acid sequence WMCPP and which is capable of binding to and inhibiting myostatin;
a splice variant of myostatin;
a regulator of the myostatin pathway; and
an antisense polynucleotide, RNAi, siRNA or an anti-myostatin ribozyme capable of inhibiting myostatin activity by inhibiting myostatin gene expression.

31. At least one myostatin antagonist as claimed in claim 30, comprising a dominant negative of myostatin selected from the Piedmontese allele and mature myostatin peptides having a C-terminal truncation at a position at of between amino acid positions 335 to 375.

32. At least one myostatin antagonist as claimed in claim 31, comprising a mature myostatin peptide having a C-terminal truncation at amino acid position 335 or 350.

33. At least one myostatin antagonist as claimed in claim 30, comprising a splice variant of myostatin selected from a polypeptide of SEQ ID NOS: 8-14, or a functional fragment or variant thereof, or a sequence having 95%, 90%, 85%, 80%, 75% or 70% sequence identify thereto.

34. At least one myostatin antagonist as claimed in claim 30 comprising a regulator of the myostatin pathway comprising the “mighty” peptide of SEQ ID NO: 16 or SEQ ID NO: 18, or a functional fragment or variant thereof, or a sequence having at least 95%, 90%, 85%, 80%, 75%, or 70% sequence identify thereto.

35. At least one myostatin antagonist as claimed in claim 29 in combination with one or more additional muscle growth promoting compounds selected from the group consisting of HGF, FGF, IGF, MGF and growth hormone for separate, sequential or simultaneous administration with the at least one myostatin antagonist to further improve muscle regeneration.

36. At least one myostatin antagonists as claimed in claim 29, formulated for local or systemic administration.

37. At least one myostatin antagonist as claimed in claim 36, formulated for oral, intravenous, cutaneous, subcutaneous, intradermal, nasal, pulmonary, intramuscular or intraperitional administration.


SEQUENCE LISTING (18 sequences)

SEQ ID NO: 1 - DNA, 966 bp, Ovine
atgcaaaaactgcaaatctttgtttatatttacctatttatgctgcttgttgctggcccagtggatctgaatgagaacagcgagcagaaggaaaatgtggaaaaaaaggggctgtgtaatgcatgcttgtggagacaaaacaataaatcctcaagactagaagccataaaaatccaaatcctcagtaagcttcgcctggaaacagctcctaacatcagcaaagatgctataagacaacttttgcccaaggctcctccactccgggaactgattgatcagtacgatgtccagagagatgacagcagcgacggctccttggaagacgatgactaccacgttacgacggaaacggtcattaccatgcccacggagtctgatcttctagcagaagtgcaagaaaaacccaaatgttgcttctttaaatttagctctaaaatacaacacaataaagtagtaaaggcccaactgtggatatatctgagacctgtcaagactcctacaacagtgtttgtgcaaatcctgagactcatcaaacccatgaaagacggtacaaggtatactggaatccgatctctgaaacttgacatgaacccaggcactggtatttggcagagcattgatgtgaagacagtgttgcaaaactggctcaaacaacctgaatccaacttaggcattgaaatcaaagctttagatgagaatggtcatgatcttgctgtaaccttcccagaaccaggagaagaaggactgaatccttttttagaagtcaaggtgcatttttacactcctccctatgggcaatggattttccataaagaaagaaaaatcatttttctagaggtctacattcaattctgtagcatacttggagaagctgtgtttaaaaggcagtcaaaaagtattcatttttgtcaaaatttcaaaattatagcctgcctttgcaatactgcagcttttaggatgaaataa

SEQ ID NO: 2 - DNA, 195 bp, Ovine
gtgcatttttacactcctccctatgggcaatggattttccataaagaaagaaaaatcatttttctagaggtctacattcaattctgtagcatacttggagaagctgtgtttaaaaggcagtcaaaaagtattcatttttgtcaaaatttcaaaattatagcctgcctttgcaatactgcagcttttaggatgaaa

SEQ ID NO: 3 - DNA, 141 bp, Ovine
atcatttttctagaggtctacattcaattctgtagcatacttggagaagctgtgtttaaaaggcagtcaaaaagtattcatttttgtcaaaatttcaaaattatagcctgcctttgcaatactgcagcttttaggatgaaa

SEQ ID NO: 4 - DNA, 966 bp, Bovine
atgcaaaaactgcaaatctctgtttatatttacctatttatgctgattgttgctggcccagtggatctgaatgagaacagcgagcagaaggaaaatgtggaaaaagaggggctgtgtaatgcatgtttgtggagggaaaacactacatcctcaagactagaagccataaaaatccaaatcctcagtaaacttcgcctggaaacagctcctaacatcagcaaagatgctatcagacaacttttgcccaaggctcctccactcctggaactgattgatcagttcgatgtccagagagatgccagcagtgacggctccttggaagacgatgactaccacgccaggacggaaacggtcattaccatgcccacggagtctgatcttctaacgcaagtggaaggaaaacccaaatgttgtttctttaaatttagctctaagatacaatacaataaactagtaaaggcccaactgtggatatatctgaggcctgtcaagactcctgcgacagtgtttgtgcaaatcctgagactcatcaaacccatgaaagacggtacaaggtatactggaatccgatctctgaaacttgacatgaacccaggcactggtatttggcagagcattgatgtgaagacagtgttgcagaactggctcaaacaacctgaatccaacttaggcattgaaatcaaagctttagatgagaatggccatgatcttgctgtaaccttcccagaaccaggagaagatggactgactccttttttagaagtcaaggtgcattttcacactcctccctatgggcaatggatgttctatagagaaagaaaactcattttcctagaggtctacattcaattctgtagcatacttggagaagctgcattgaaaaggcagtcaaaaagtattcattttggtcaaaatttcaaaattatagcctgcctttgcaatactgcagcttttaggatgaaataa

SEQ ID NO: 5 - DNA, 195 bp, Bovine
gtgcattttcacactcctccctatgggcaatggatgttctatagagaaagaaaactcattttcctagaggtctacattcaattctgtagcatacttggagaagctgcattgaaaaggcagtcaaaaagtattcattttggtcaaaatttcaaaattatagcctgcctttgcaatactgcagcttttaggatgaaa

SEQ ID NO: 6 - DNA, 141 bp, Bovine
ctcattttcctagaggtctacattcaattctgtagcatacttggagaagctgcattgaaaaggcagtcaaaaagtattcattttggtcaaaatttcaaaattatagcctgcctttgcaatactgcagcttttaggatgaaa

SEQ ID NO: 7 - DNA, 945 bp, Belgian Blue
atgcaaaaactgcaaatctctgtttatatttacctatttatgctgattgttgctggcccagtggatctgaatgagaacagcgagcagaaggaaaatgtggaaaaagaggggctgtgtaatgcatgtttgtggagggaaaacactacatcctcaagactagaagccataaaaatccaaatcctcagtaaacttcgcctggaaacagctcctaacatcagcaaagatgctatcagacaacttttgcccaaggctcctccactcctggaactgattgatcagttcgatgtccagagagatgccagcagtgacggctccttggaagacgatgactaccacgccaggacggaaacggtcattaccatgcccacggagtctgatcttctaacgcaagtggaaggaaaacccaaatgttgtttctttaaatttagctctaagatacaatacaataaactagtaaaggcccaactgtggatatatctgaggcctgtcaagactcctgcgacagtgtttgtgcaaatcctgagactcatcaaacccatgaaagacggtacaaggtatactggaatccgatctctgaaacttgacatgaacccaggcactggtatttggcagagcattgatgtgaagacagtgttgcagaactggctcaaacaacctgaatccaacttaggcattgaaatcaaagctttagatgagaatggccatgatcttgctgtaaccttcccagaaccaggagaagatggactggtgcattttcacactcctccctatgggcaatggatgttctatagagaaagaaaactcattttcctagaggtctacattcaattctgtagcatacttggagaagctgcattgaaaaggcagtcaaaaagtattcattttggtcaaaatttcaaaattatagcctgcctttgcaatactgcagcttttaggatgaaataa

SEQ ID NO: 8 - protein, 321 aa, Ovine
MQKLQIFVYIYLFMLLVAGPVDLNENSEQKENVEKKGLCNACLWRQNNKSSRLEAIKIQILSKLRLETAPNISKDAIRQLLPKAPPLRELIDQYDVQRDDSSDGSLEDDDYHVTTETVITMPTESDLLAEVQEKPKCCFFKFSSKIQHNKVVKAQLWIYLRPVKTPTTVFVQILRLIKPMKDGTRYTGIRSLKLDMNPGTGIWQSIDVKTVLQNWLKQPESNLGIEIKALDENGHDLAVTFPEPGEEGLNPFLEVKVHFYTPPYGQWIFHKERKIIFLEVYIQFCSILGEAVFKRQSKSIHFCQNFKIIACLCNTAAFRMK

SEQ ID NO: 9 - protein, 65 aa, Ovine
VHFYTPPYGQWIFHKERKIIFLEVYIQFCSILGEAVFKRQSKSIHFCQNFKIIACLCNTAAFRMK

SEQ ID NO: 10 - protein, 47 aa, Ovine
IIFLEVYIQFCSILGEAVFKRQSKSIHFCQNFKIIACLCNTAAFRMK

SEQ ID NO: 11 - protein, 321 aa, Bovine
MQKLQISVYIYLFMLIVAGPVDLNENSEQKENVEKEGLCNACLWRENTTSSRLEAIKIQILSKLRLETAPNISKDAIRQLLPKAPPLLELIDQFDVQRDASSDGSLEDDDYHARTETVITMPTESDLLTQVEGKPKCCFFKFSSKIQYNKLVKAQLWIYLRPVKTPATVFVQILRLIKPMKDGTRYTGIRSLKLDMNPGTGIWQSIDVKTVLQNWLKQPESNLGIEIKALDENGHDLAVTFPEPGEDGLTPFLEVKVHFHTPPYGQWMFYRERKLILLEVYIQFCSILGEAALKRQSKSIHFGQNFKIIACLCNTAAFRMK

SEQ ID NO: 12 - protein, 65 aa, Bovine
VHFHTPPYGQWMFYRERKLILLEVYIQFCSILGEAALKRQSKSIHFGQNFKIIACLCNTAAFRMK

SEQ ID NO: 13 - protein, 47 aa, Bovine
LILLEVYIQFCSILGEAALKRQSKSIHFGQNFKIIACLCNTAAFRMK

SEQ ID NO: 14 - protein, 314 aa, Belgian Blue
MQKLQISVYIYLFMLIVAGPVDLNENSEQKENVEKEGLCNACLWRENTTSSRLEAIKIQILSKLRLETAPNISKDAIRQLLPKAPPLLELIDQFDVQRDASSDGSLEDDDYHARTETVITMPTESDLLTQVEGKPKCCFFKFSSKIQYNKLVKAQLWIYLRPVKTPATVFVQILRLIKPMKDGTRYTGIRSLKLDMNPGTGIWQSIDVKTVLQNWLKQPESNLGIEIKALDENGHDLAVTFPEPGEDGLVHFHTPPYGQWMFYRERKLILLEVYIQFCSILGEAALKRQSKSIHFGQNFKIIACLCNTAAFRMK

SEQ ID NO: 15 - DNA, 576 bp, Ovine
atggcgtgcggggcgacactgaagcggcccatggagttcgaggcggcgctgctgagccctggctctccgaagcggcggcgctgcgcccctctgtccggccccactccgggcctcaggcccccggacgccgaaccgccgccgctgcttcagacgcagaccccaccgccgactctgcagcagcccgccccgcccggcagcgagcggcgccttccaactccggagcaaatttttcagaacataaaacaagaatatagtcgttatcagaggtggagacatttagaagttgttcttaatcagagtgaagcttgtacttcggaaagtcagcctcactcctcagcactcacagcacctagttctccaggttcctcctggatgaaaaaggaccagcccacctttaccctccgacaagttggaataatatgtgagcgtctcttaaaagactatgaagataaaattcgggaggaatatgagcaaatcctcaatactaaactagcagaacaatatgaatcttttgtgaaattcacacatgatcagattatgcgacgatatgggacaaggccaacaagctatgtatcc

SEQ ID NO: 16 - protein, 192 aa, Ovine
MACGATLKRPMEFEAALLSPGSPKRRRCAPLSGPTPGLRPPDAEPPPLLQTQTPPPTLQQPAPPGSERRLPTPEQIFQNIKQEYSRYQRWRHLEVVLNQSEACTSESQPHSSALTAPSSPGSSWMKKDQPTFTLRQVGIICERLLKDYEDKIREEYEQILNTKLAEQYESFVKFTHDQIMRRYGTRPTSYVS

SEQ ID NO: 17 - DNA, 576 bp, Bovine
atggcgtgcggggcgacactgaagcggcccatggagttcgaggcggcgctgctgagccctggctctccgaagcgacggcgctgcgcccctctgtccggccccactccgggcctcaggcccccggacgccgaaccgccaccgctgcttcagacgcagatcccaccgccgactctgcagcagcccgccccgcccggcagcgaccggcgccttccaactccggagcaaatttttcagaacataaaacaagaatatagtcgttatcagaggtggagacatttagaagttgttcttaatcagagtgaagcttgtacttcggaaagtcagcctcactcctcaacactcacagcacctagttctccaggttcctcctggatgaaaaaggaccagcccacctttacgctccgacaagttggaataatatgtgagcgtctcttaaaagactatgaagataaaattcgggaggaatatgagcaaatcctcaatactaaactagcagaacaatatgaatcttttgtgaaattcacacatgatcagattatgcgacgatatgggacaaggccaacaagctatgtatcc

SEQ ID NO: 18 - protein, 192 aa, Bovine
MACGATLKRPMEFEAALLSPGSPKRRRCAPLSGPTPGLRPPDAEPPPLLQTQIPPPTLQQPAPPGSDRRLPTPEQIFQNIKQEYSRYQRWRHLEVVLNQSEACTSESQPHSSTLTAPSSPGSSWMKKDQPTFTLRQVGIICERLLKDYEDKIREEYEQILNTKLAEQYESFVKFTHDQIMRRYGTRPTSYVS